(19) United States
(12) Patent Application Publication
(10) Pub. No.: US 2025/0242157 A1
(43) Pub. Date: Jul. 31, 2025

(54) SYSTEMS AND METHODS USING MACHINE LEARNING FOR PHRENIC NERVE MONITORING AND MODULATION

(71) Applicant: LUNAIR MEDICAL, Minneapolis, MN (US)

(72) Inventors: Orhan SOYKAN, Lino Lakes, MN (US); Mark CHRISTOPHERSON, Shoreview, MN (US)

(21) Appl. No.: 19/041,548

(22) Filed: Jan. 30, 2025

Related U.S. Application Data
(60) Provisional application No. 63/627,023, filed on Jan. 30, 2024.

(51) Int. Cl. A61N 1/36

(52) U.S. Cl. CPC A61N 1/36014; A61N 1/3601

(57) ABSTRACT

A system for determining parameters for implantable devices that are designed to electrically stimulate the phrenic nerve to treat sleep disordered breathing is provided. The system may use techniques to automate the identification of the parameters that are needed for the recognition and classification of the input signals as well as their values. The system may use techniques for the optimization of the electrical stimulation therapy that is delivered by the implantable device.

### CROSS REFERENCE(S) TO RELATED APPLICATION(S)

This application claims priority to U.S. Provisional Patent Application 63/627,023, filed Jan. 30, 2024, the entire contents of which are hereby incorporated by reference.

### TECHNICAL OVERVIEW

The techniques herein relate to the methods for determining programming parameters for implantable devices that are designed to electrically stimulate the phrenic nerve to treat sleep disordered breathing—such as airway collapse in patients with Obstructive Sleep Apnea (OSA). The techniques may be used to automate the identification of parameters that are needed for the recognition and classification of the input signals as well as their values. Similarly, techniques disclosed are also used for the optimization of the electrical stimulation therapy delivered by the implantable device.

### INTRODUCTION

Healthy Sleep is an important part of our lives. It improves physical and mental health. Sleep happens in stages, including REM sleep and non-REM sleep. When humans sleep, their body has a chance to rest and restore energy. A good night's sleep can help us cope with stress, solve problems, or recover from illness. Not getting enough sleep can lead to many health concerns, affecting how we think and feel.

During sleep, a person usually passes through four sleep stages: non-REM N1, N2, N3, and REM (rapid eye movement). These stages of sleep progress in a cycle from N1 to REM sleep, then the cycle starts over again with N1 or N2. Healthy children and adults spend almost 50 percent of their total sleep time in N2 sleep, about 20 percent in REM sleep, and the remaining 30 percent in the other stages.

During N1, which is light sleep, we drift in and out of sleep and can be awoken easily. Our eyes move very slowly, and muscle activity slows. People awakened from N1 sleep often remember fragmented visual images.

When we enter N2 sleep, our eye movements stop and our brain waves (fluctuations of electrical activity that can be measured by electroencephalogram electrodes (EEG)) become slower, with occasional bursts of rapid waves called sleep spindles.

In N3, extremely slow brain waves called delta waves begin to appear, interspersed with smaller, faster waves until delta waves occur almost exclusively. It is very difficult to wake someone during N3, which is also called deep or slow wave sleep.

When we switch into REM sleep, our breathing becomes more rapid, irregular, and shallow, our eyes jerk rapidly in various directions, and our limb muscles become temporarily paralyzed during sleep. Our heart rate increases, our blood pressure rises. When people wake up during REM sleep, they often describe dreams.

The first REM sleep period usually occurs about 70 to 90 minutes after we fall asleep. A complete sleep cycle takes 90 to 110 minutes on average. The first sleep cycles each night contain relatively short REM periods and long periods of deep sleep. As the night progresses, REM sleep periods increase in length while deep sleep decreases. By morning, healthy people spend nearly all their sleep time in stages 1, 2, and REM.

Although neurophysiology of sleep may not be completely understood, it can be recognized that a good night of sleep is a night of continuous, uninterrupted sleep that cycles through sleep stages, including REM. Sleep disorders, specifically those under the category of sleep apnea syndrome, frequently interrupt these continuous sleep patterns and lead to daytime sleepiness, fatigue and have many other serious deleterious effects on both mental and physical health.

Sleep disordered breathing is a common sleep disorder where patients have repetitive episodes of either cessation of breathing (apneas) or periods of reduced flow (hypopneas) during sleep. For patients with sleep disordered breathing, sleep is interrupted with 10 or more second periods without proper airflow, occurring hundreds of times during a typical night's sleep. Apneas, generally originate as either obstructive, central, or some combination of the two etiologies.

Obstructive Sleep Apnea (OSA) is a well-recognized disease that affects millions of people. It is a form of sleep disordered breathing characterized by periodic interruptions of lung ventilation that disrupt sleep due to a momentary collapse and obstruction of the pharyngeal airway. Obstruction of the pharyngeal airway can be attributed to decreased upper airway muscle tone, relaxing muscles that support the soft tissues in the throat, such as the tongue and/or soft palate. Relaxation of these muscles results in the narrowing of the pharyngeal airway, causing airflow obstruction thus limiting airflow and leading to a decrease in oxygen saturation. Central Sleep Apnea (CSA) is a less common sleep disorder that is characterized by apneas due to a lack of signals from the respiratory center. With CSA, thoracic neural receptors fail to send a signal to the respiratory center to initiate inspiration. As a result, airflow ceases due to no respiratory muscle activity. Mixed Sleep Apnea is a combination of OSA and CSA where there is both decreased respiratory drive and decreased upper airway muscle tone.

Pathogenesis of the Upper Airway (UA) obstruction during sleep is due to (a) a primary sleep-related loss of UA neuromotor tone and (b) a lack of adequate compensatory reflex responses that mitigate the obstruction. The inventors believe that OSA may be caused by an inadequate reflex mechanism in response to an obstructed airway.

### Control of Respiration:

In healthy individuals, upper airway stability during sleep is ensured by coordinated and synchronized central control of the respiratory system, specifically the airway muscles, that are comprised of about twenty airway dilator and constrictor muscles. The central nervous system (CNS) pattern generator, also referred to as the respiratory control system, in the medulla of the brain receives inputs from physiologic sensors (also called receptors) via various afferent sensory nerve fibers and controls airway muscles via efferent motor fibers. These physiologic sensors provide physiologic feedback used by the medulla to trigger a reflex from the effectors in a closed loop reflex arrangement. These reflexes are known as “autonomic” since they do not depend on consciousness. In some cases, the reflexes become insufficient for optimal health during sleep.

Respiration during sleep is governed mainly by three systems. As illustrated in FIG. 13, the Central Neural Controller, the respiratory system itself, and the cardiovascular system. The brainstem, cortex, limbic system, and hypothalamus primarily contribute to respiratory effort from the central neural controller. The central pattern generator from the brainstem controls the periodic nature of inspiration and expiration. Three main groups of neurons located in the pons and medulla aid in generating rhythmic breathing: the medullary respiratory center, apneustic center, and pneumotaxic center. The medulla respiratory center is comprised of different groups of cells that are responsible for the basic rhythm of ventilation, including the generation of respiratory rhythm, inspiration, and expiration. These cells generate repetitive bursts of action potentials without afferent stimuli to send nervous impulses to the diaphragm and other inspiratory muscles. The rhythmic pattern of inspiration begins with an initialization of several seconds where no activity occurs. Action potentials then occur to create a crescendo for a period of several seconds, causing inspiratory muscle activity to become stronger. The inspiratory action potentials then cease, and inspiratory muscle tone falls to its preinspiratory level. The apneustic center creates impulses that have an excitatory effect on the inspiratory area of the medulla, prolonging the action potentials, causing abnormal breathing. The pneumotaxic center regulates inspiration volume and respiration rate by inhibiting inspiration. However, a normal breathing pattern can exist without the pneumotaxic center, leading scientists to believe that this center's role is for “fine-tuning” the respiratory rhythm.

The cortex may override the function of the brainstem in certain situations, such as hyperventilation or voluntary hypoventilation. Other parts of the brain, such as the limbic system and hypothalamic, can alter rhythmic breathing as well, due to different emotional states.

Sensory inputs to the respiratory center include signals from chemoreceptors and many distributed mechanoreceptors. Central chemoreceptors are involved with the minute-by-minute control of ventilation and react to the amount of CO₂ dissolved in the blood (PCO₂), but not the amount of oxygen (PO₂). In additional, central chemoreceptors respond to changes in hydrogen ion (H⁺) concentrations, where an increase in H⁺ concentration stimulates ventilation, and a decrease inhibits ventilation. Peripheral chemoreceptors are also a type of chemoreceptor that aid in ventilation and are important for maintaining homeostasis during hypoxemia. Peripheral chemoreceptors respond to a decrease in arterial PO₂, an increase in PCO₂, a change in H⁺, and a change in arterial pH.

Afferent receptors in the tracheobronchial tree and lungs detect alterations in airway pressure, temperature, air flow, and lung stretch which may be indicators of a collapsed airway. The afferent receptors provide feedback signals to the spinal cord or CNS which may respond to the feedback signals by triggering reflex responses that stimulate the upper airway muscles, which can then mitigate airway obstruction.

Some of the afferent receptors that aid in ventilation and respiration are mechanoreceptors. Lung receptors are one type of afferent receptor that provide inputs carried via the vagus nerve to the CNS to influence ventilation. Pulmonary stretch receptors are a type of lung receptor located in the smooth muscle of the airway walls that respond to changes in lung inflation. That is, these stretch receptors contribute to switching off inspiration and initiate exhalation based on how inflated the lungs are. Feedback from these stretch receptors inhibit further inspiratory muscle activity as the lungs inflate, and an initiation of inspiratory activity results in a deflation of the lungs.

Other types of lung receptors include irritant receptors, J-receptors, and bronchial C fibers. Irritant receptors are stimulated by inhaled noxious stimulus such as cigarette smoke or inhaled dust. These receptors are more rapidly adapting than stretch receptors and result in tachypnea. J-receptors also cause tachypnea, dyspnea, and apnea, as a result of events such as pulmonary edema, pulmonary emboli, pneumonia, etc. Bronchial C fibers are supplied by bronchial circulation rather than pulmonary circulation and respond to chemicals injected into bronchial circulation. Stimulation of the bronchial C fibers also results in tachypnea. FIG. 14 shows the various sensors and their afferent nerves that carry the information regarding the respiration and ventilation to the central nervous system (CNS). Afferent and efferent pathways with CNS involvement are shown as a block diagram in FIG. 12.

Additional receptors also can impact ventilation and respiration. These include receptors in the nose, nasopharynx, larynx, trachea. These receptors respond to, for example, mechanical and chemical stimulation—e.g., irritants. Joint and muscle receptors impact ventilation by sending signals during exercise. Receptors in the intercostal muscles and diaphragm contain muscle spindles that sense elongation of the muscle. These receptors adjust the output of respiratory muscles if the degree of muscular work has not been met or has been exceeded, helping to control the strength and degree of contraction. When unusually large respiratory efforts are required to move the lung and chest wall, dyspnea occurs due to the discrepancy between the output from the CNS controller and the amount of stretch sensed by these receptors. Arterial baroreceptors can cause reflex hypoventilation or apnea through stimulation of the aortic and carotid sinus baroreceptors. Accordingly, many afferent nerves can induce changes in ventilation.

### Sensory (Afferent) Nerves:

Sensory nerves, also known as afferents, carry information from the peripheral organs to the central nervous system. They respond to the sensory stimulation by changing their firing rate, as illustrated in FIG. 04. In the example that is shown in FIG. 03, the firing frequency of the sensory nerve begins to increase, in this case from zero, as the input stimulus is increased beyond P_{MIN}. The nerve firing rate saturates at f_{MAX} once the stimulus level reaches and exceeds P_{MAX}. It should be noted that some sensory nerves maintain a non-zero firing rate even in the absence of any physical stimuli.

In addition to having a non-linear input-output relationship as shown in FIG. 04, sensory nerves also have a time dependent response to the physical stimulus that they receive. Although they produce a rapid response to the initial stimulus, their response to the same level of stimulus does decrease over time. This phenomenon is further illustrated in FIG. 05 with the use of a model of the sensory nerve.

As shown in FIG. 05, the output of the sensory nerve is its firing frequency, f_{OUT}, which is determined as the sum of two signals. The first signal contributing to f_{OUT} comes from the lower pathway that is shown in FIG. 05 and it is the product of input stimulus, P_{IN}, and K_{P} 0540, where K_{P} represents a proportionality constant. The second signal contributing to f_{OUT} is produced by the upper pathway shown in FIG. 05 and it is a result of the changes in the input stimulus P_{IN}. K_{D} 0510 represents a proportionality constant for the rate of change related signal contributing to f_{OUT}. Remaining two items in the upper pathway are the rate of change determining differentiator 0520 and positive only determinant 0530. The presence of the last item, the positive only determinant, indicates that the rate of change response that is described earlier applies only to the onset of the physical stimulation, and is absent at the conclusion of the physical stimulation. Above discussion illustrates the need for a signal processor that can help interpret the signals that are recorded from a sensory nerve.

### Motor (Efferent) Nerves:

Motor nerves, also known as efferents, carry information from the central nervous system to peripheral organs. They provide the excitation to the muscles, such as the diaphragm of the respiratory system. A typical firing pattern of a motor neuron is shown in FIG. 03. A normal firing duration of the motor neuron is usually 300 milli-seconds, as shown by the train of firings 0310 from t=100 milli-seconds to t=400 milli-seconds in FIG. 03. It should be noted that the firing frequency of the motor neuron 0340 is not fixed, but it increases as a function of time. In the example that is shown in FIG. 04, the firing frequency 0340 increases from 40 Hertz to 60 Hertz, which may or may not be in a linear fashion. Most of the time, the recorded signal is too noisy to be analyzed in detail, hence its time integral 0320 is constructed. Furthermore, a smoothed version 0330 of the time integral is used for signal processing purposes.

Since the motor neurons carry excitation signals to the nerves, they tend to have larger cross sections compared to the sensory neurons. However, the distribution of cross-sectional areas of motor axons in the phrenic nerve changes as a function of aging, as illustrated in FIG. 08. As the individual ages, the distribution shifts toward the motor neurons with a smaller cross-sectional area. This fact illustrates the need for the signal processors to be customized for the physiology of the patient who is wearing the device.

### Electrical Stimulation of Nerves:

Both the motor nerves and the sensory nerves can be stimulated with externally applied electrical signals. This can be done with various types of electrodes, such as the cuff electrode as shown in FIG. 10. Typical strength-duration curves for the capture of sensory and motor nerves are shown in FIG. 07. Due to reduced cross sectional area of the sensory nerves, larger pulse-widths are required to capture them compared to the motor nerves. Following the delivery of the electrical stimulation to the nerve, a resulting action potential travels through the nerve fiber, as shown in FIG. 09. It should be noted that even though the duration of the action potential waveform that is shown in FIG. 09 is rather short, muscle contractions that last much longer can be achieved by repeated application of the stimulation train to generate a sustained tetanic contraction.

Waveforms that are used for the electrical stimulation of nerves are shown in FIG. 15, where FIG. 15A shows a monopolar stimulation waveform and the FIG. 15B shows a bipolar stimulation waveform. Following is list of parameters of the stimulation waveform that are programmable: Amplitude 1520 and 1550; Pulse-width 1530 and 1570; Pulse period 1510 and 1580; Train duration 1540 and 1590; Silence duration 1545 and 1595.

Furthermore, the stimulation can be delivered as a voltage or a current waveform, and may or may not be constant during the actual stimulation duration, e.g., pulse-width.

Accordingly, it will be appreciated that new and improved techniques, systems, and processes are continually sought after.

### SUMMARY

In certain example embodiments, a device (e.g., a medical device) is provided that can be used to determine the optimal parameters for detection of respiratory activity and best modality for the modulation of the nervous system in response. Related systems, methods for the building of the device, as well as the design parameters are provided.

In certain example embodiments hardware and/or software is used to optimize one or more algorithms that may then be deployed and used in an implantable device. In certain example embodiments, the device is defined such that it can be used online or offline. In certain examples, an ongoing optimization process may be provided to take place once, periodically, or when needed.

Additional features further allow the device to work with different patients, different sensing and stimulation configurations, and with different modes of connection to the implantable device.

Other objects, features, and advantages will become apparent to those skilled in the art from the following detailed description. It is to be understood, however, that the detailed description and specific examples, while indicating some embodiments, are given by way of illustration and not limitation. Many changes and modifications within the scope of the present invention may be made without departing from the teachings of the present invention.

### BRIEF DESCRIPTION OF THE DRAWINGS

These and other features and advantages will be better and more completely understood by referring to the following detailed description of example non-limiting illustrative embodiments in conjunction with the drawings of which:

FIG. 01 shows the clinical configuration of the phrenic nerve monitoring and modulation system during its training phase,

FIG. 02 shows an operational block diagram of the phrenic nerve monitoring and modulation system,

FIG. 03 shows the various features of the waveform of a motor neuron while it is firing,

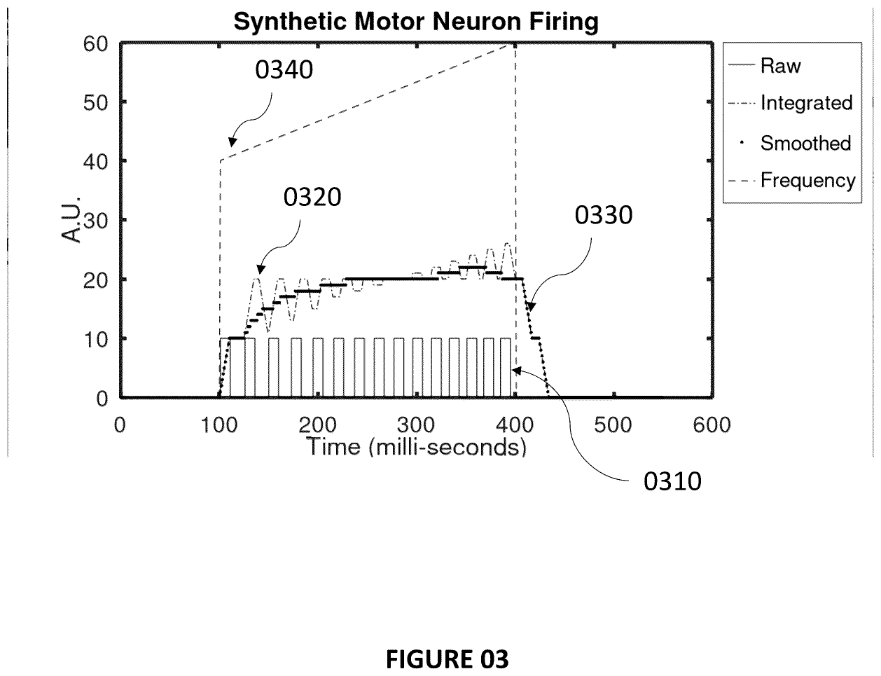

FIG. 04 shows the relationship between the firing frequency and

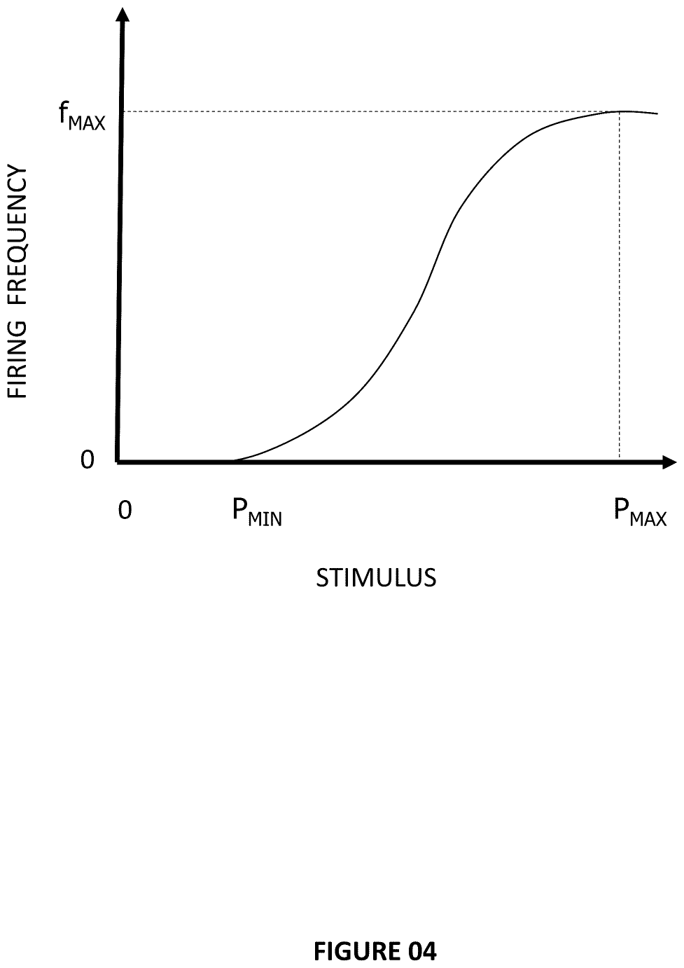

drive stimulus for a neuron,

FIG. 05 shows a simplified block diagram of a sensory neuron,

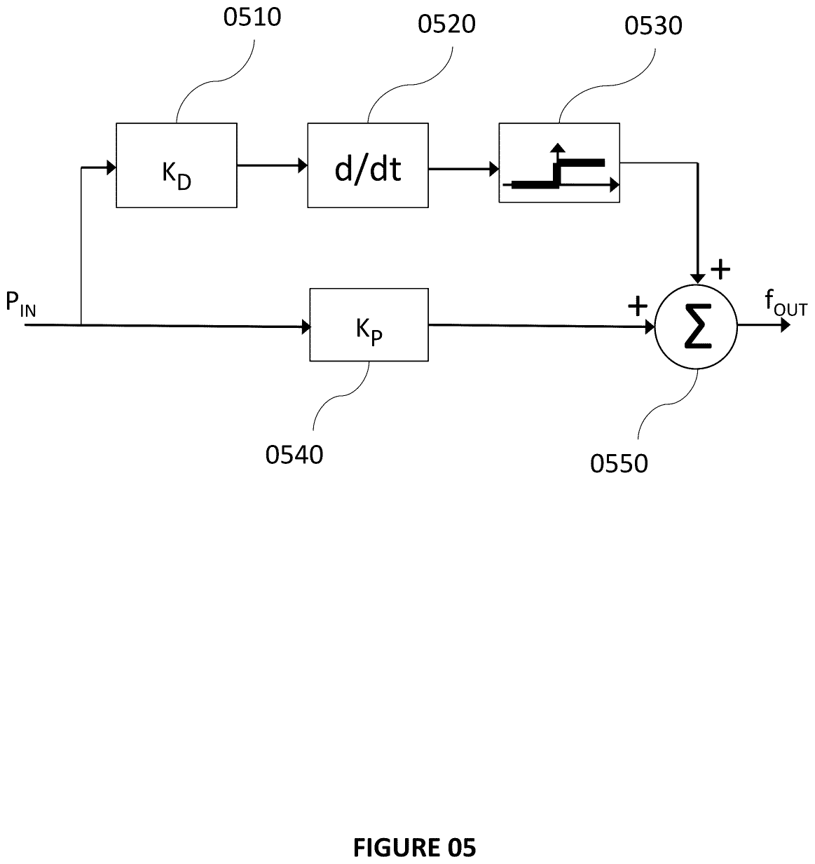

FIG. 06 shows the Shannon Plot for safe stimulation parameters,

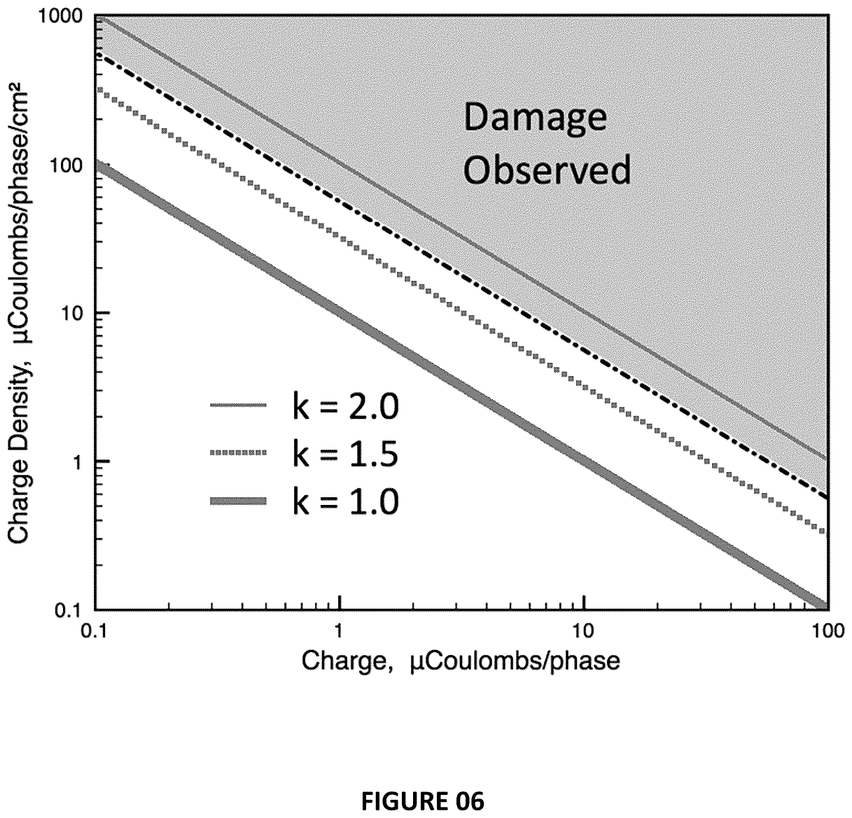

FIG. 07 shows the strength-duration curves for motor neurons,

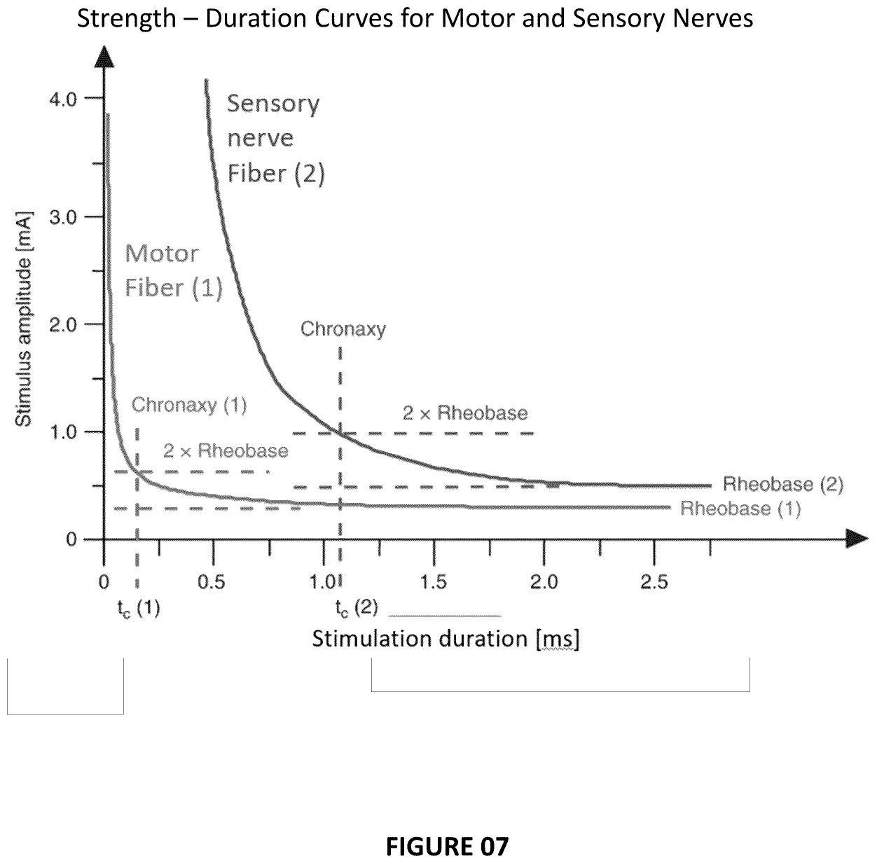

FIG. 08 shows the changes in the composition of motor axons in the phrenic nerve as a result of aging,

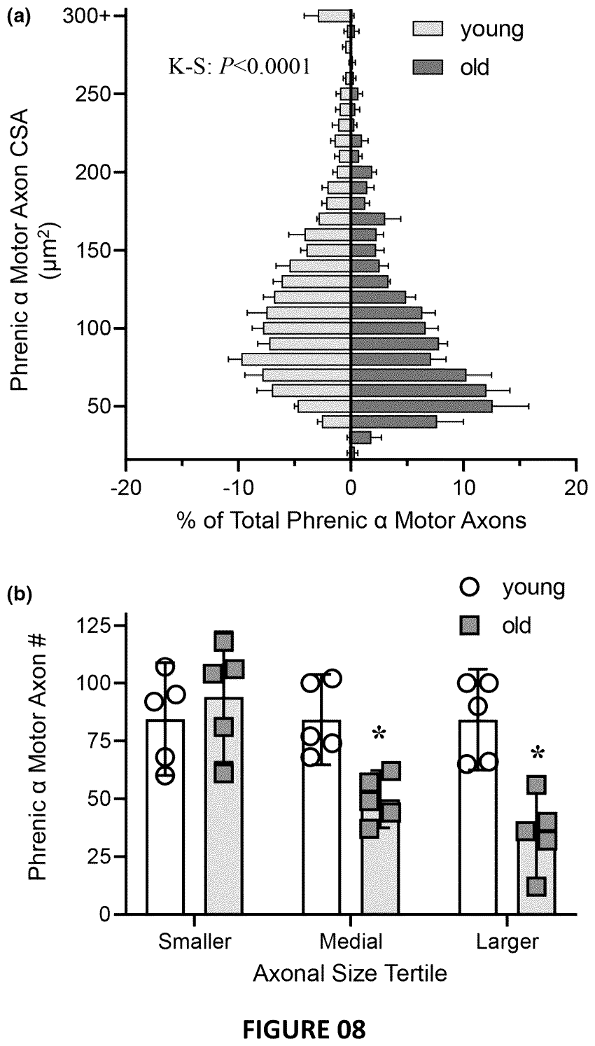

FIG. 09 shows the morphology of the electrically evoked compound action potential of a motor neuron,

FIG. 10 shows a tripolar cuff electrode for nerve stimulation,

FIG. 11 shows the relationship between the stimulation energy delivered to the phrenic nerve and the resulting contraction of the diaphragm,

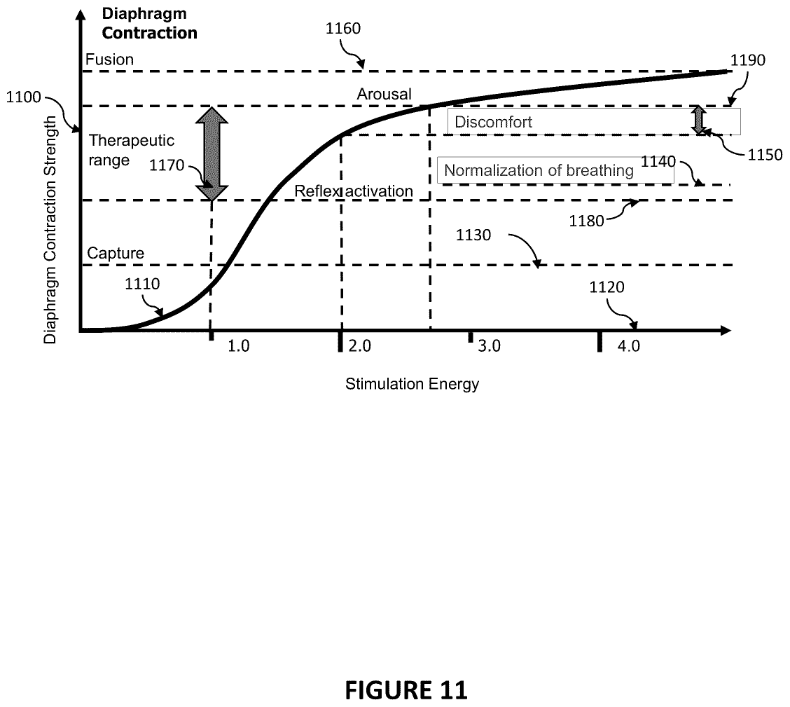

FIG. 12 shows afferent and efferent pathways with CNS involvement in the control of breathing,

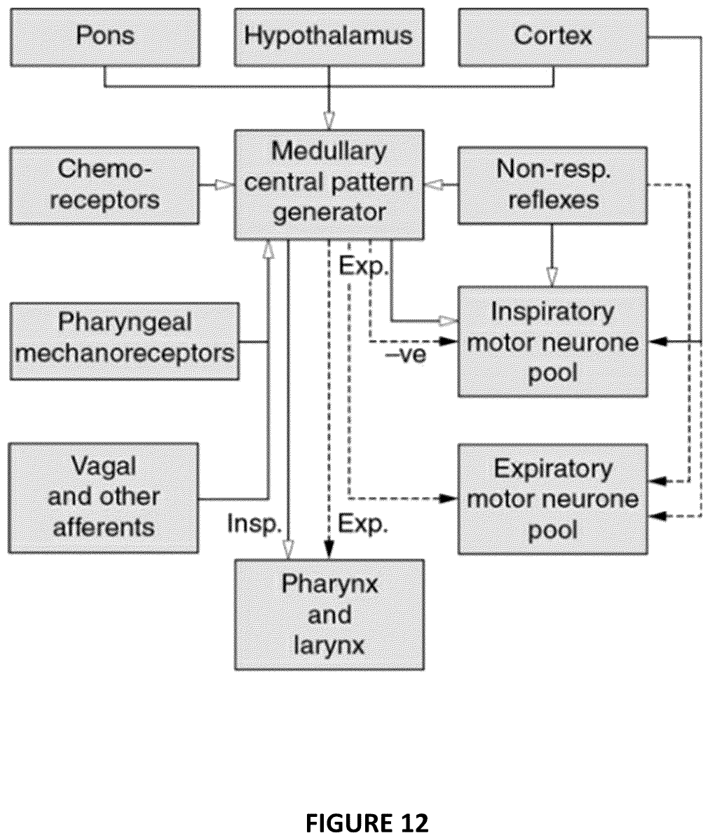

FIG. 13 shows the three systems governing the respiration during sleep,

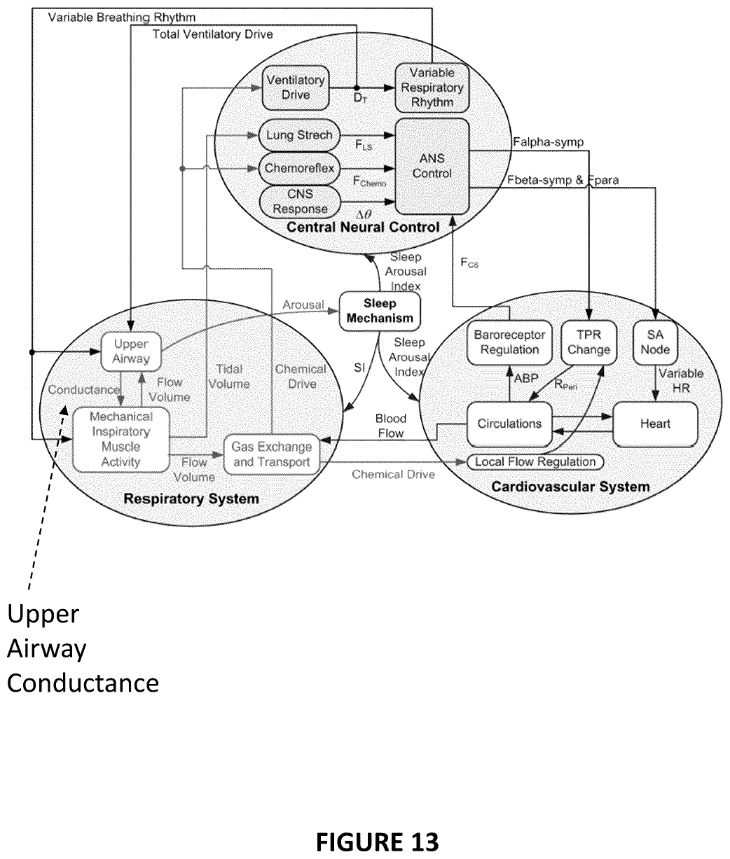

FIG. 14 shows the various sensors and their afferent nerves that carry the information regarding the respiration and ventilation to the central nervous system,

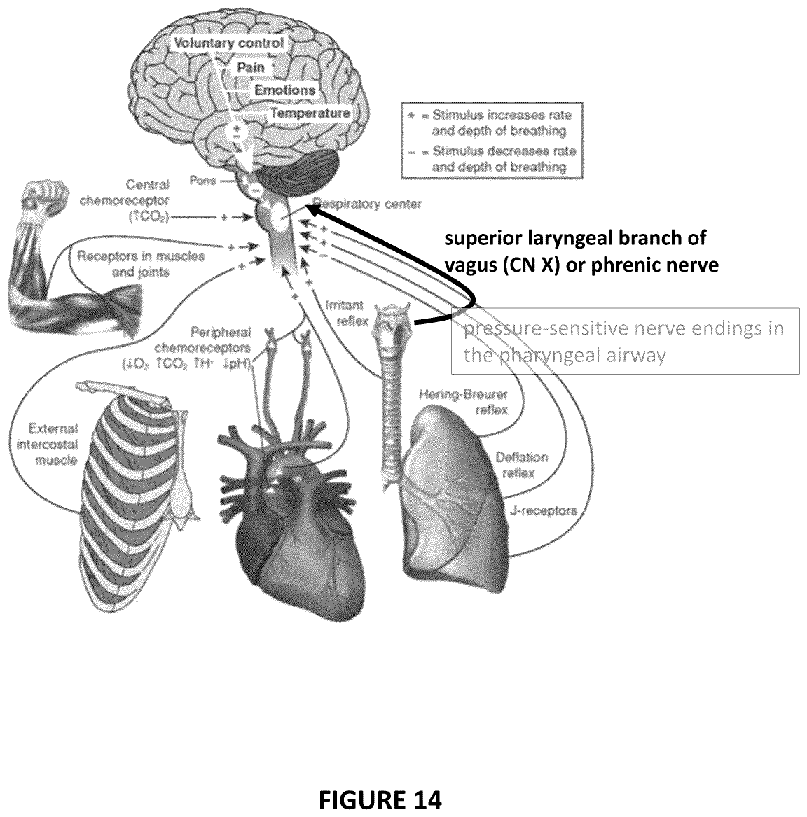

FIGS. 15A and 15B show, respectively, unipolar and bipolar stimulation waveforms and their parameters,

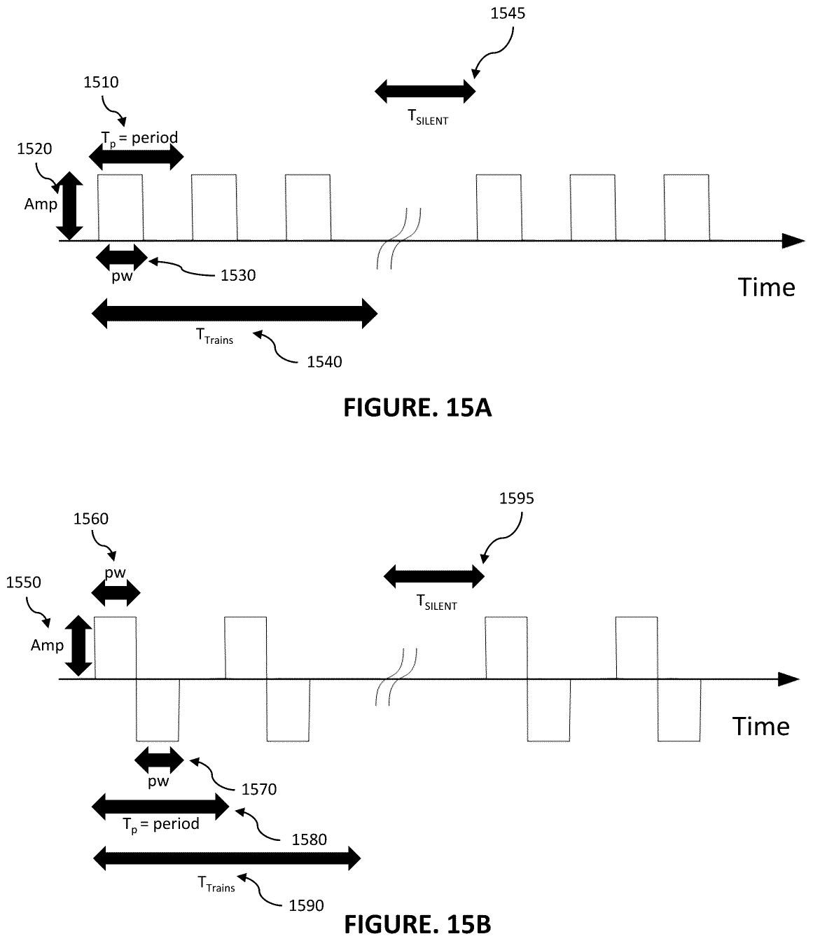

FIG. 16 shows an exemplary timing of the stimulation waveform in relation to respiration,

FIG. 17 shows an exemplary implementation of artificial neural network to be used in the input signal processor,

FIG. 18 shows the output of the same gyroscope mounted on the same location on the chests of two age and weight matched individuals, laying flat on their back and breathing at a given rate,

FIG. 19 shows an exemplary implementation of convolutional neural network to be used in the input signal processor,

FIG. 20 shows an exemplary implementation of a generative adversarial network (GAN) to be used in the input signal processor,

FIG. 21 shows an exemplary implementation of an autoencoder to be used in the input signal processor,

FIGS. 22A, 22B, and 22C show the model learning during the progression of Bayesian optimization process,

FIG. 23 shows the linear support vector machine example with complete separation of outputs,

FIG. 24 shows the linear support vector machine example with incomplete separation of outputs,

FIG. 25 shows an example of a classification and regression tree (CART).

FIG. 26 shows the configuration of output processor during the training phase,

FIG. 27 shows the configuration of implantable device during runtime,

FIG. 28 shows the steps of the implementation where the optimization is carried out in advance of the implantation,

FIG. 29 shows the steps of the implementation where the optimization is carried out after the implantation,

FIG. 30 shows the steps of the implementation where the optimization is carried out in an ongoing basis,

FIG. 31 shows an example computing device that may be used in some embodiments to implement features described herein,

FIG. 32 shows an artificial neural network (ANN) implementation for the output processor according to certain example embodiment, and

FIG. 33 shows an example GAN implementation for the output processor according to certain example embodiments.

### DETAILED DESCRIPTION

In the following description, for purposes of explanation and non-limitation, specific details are set forth, such as particular nodes, functional entities, techniques, protocols, etc. in order to provide an understanding of the described technology. It will be apparent to one skilled in the art that other embodiments may be practiced apart from the specific details described below. In other instances, detailed descriptions of well-known methods, devices, techniques, etc. are omitted so as not to obscure the description with unnecessary detail.

Sections are used herein solely in order to orient the reader as to the general subject matter of each section; as will be seen below, the description of many features spans multiple sections, and headings should not be read as affecting the meaning of the description included in any section.

### Overview

A system is provided that is used to determine parameters for detection of respiratory activity and then to cause changes in the nervous system of a patient. The techniques herein include those that relate to the treatment of sleep disordered breathing.

“Sensory nerves” and “afferents” are terms used interchangeably and they refer to nerves originating at peripheral organs such as the diaphragm and carry information to the central nervous system.

“Motor nerves” and “efferents” are terms used interchangeably and they refer to nerves originating at the central nervous system and produce excitation to the muscles such as the diaphragm.

“Cuff electrode” and “electrode” are terms used interchangeably and they refer to the device that is in contact with the target tissue, such as the phrenic nerve, while being connected to the electronics.

The term electrically evoked compound action potential (eCAP) represents the synchronous firing of a population of electrically stimulated nerve fibers. It can be directly recorded on a surgically exposed nerve trunk.

The term “electronics” refers to a set of electronic components including connectors, conductors, active components such as amplifiers, passive components such as resistors, capacitors, inductors, and crystals, digital components such as gates, timers, and microprocessors, analog components such as transistors and switches, as well as power sources such as batteries and power adapters. Furthermore, the components of the telemetry systems, such as antennas, resonators, and filters are referred to as electronics.

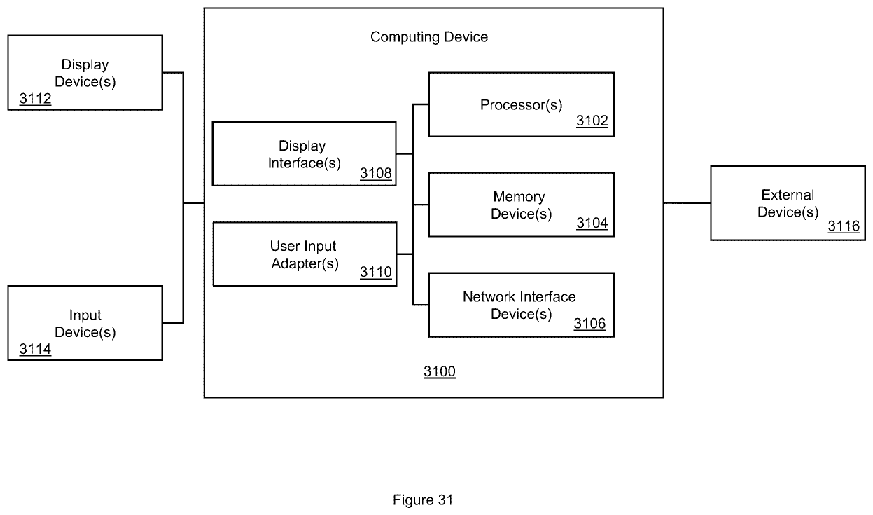

The term “processor” refers to a hardware processor that includes digital circuitry that can be configured to perform one or more tasks. For example, one or more tasks in a predetermined order based on its inputs and produces digital outputs. An example of a processor is discussed in connection with FIG. 31.

In many places in this document, software (e.g., modules, software engines, services, applications, and the like) and actions (e.g., functionality) performed by software are described. This is done for ease of description; it should be understood that, whenever it is described in this document that software performs any action, the action is in actuality performed by underlying hardware elements (such as a processor and a memory device) according to the instructions that comprise the software. Such functionality may, in some embodiments, be provided in the form of firmware and/or hardware implementations. Further details regarding this are provided below in, among other places, the description of FIG. 31.

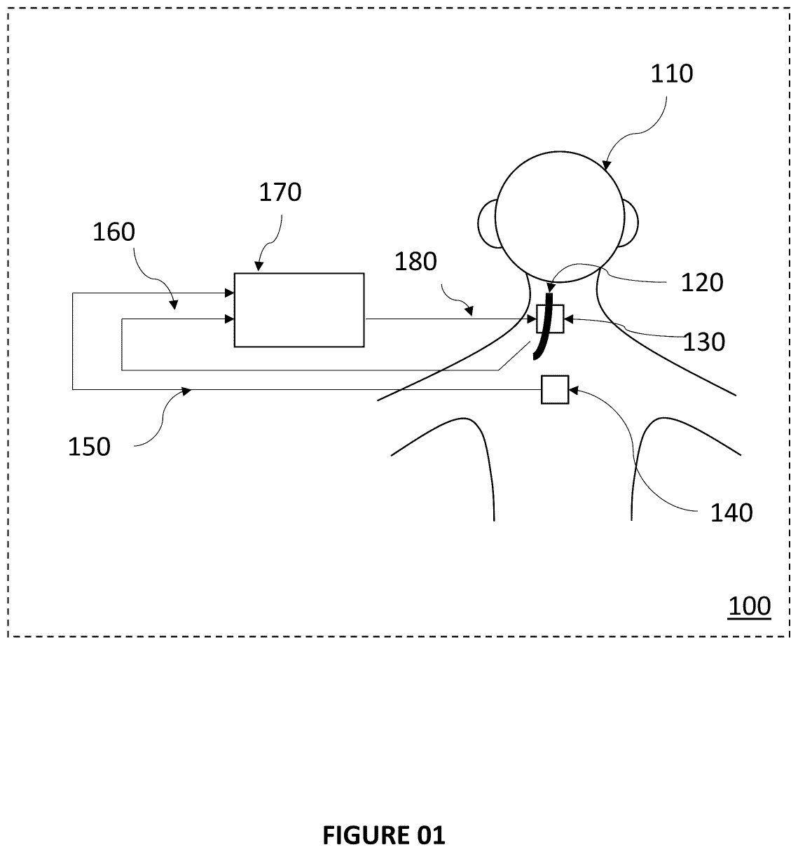

### Description of FIG. 01

FIG. 01 shows an example clinical configuration of a phrenic nerve monitoring and modulation system during its training phase according to certain example embodiments. The phrenic nerve monitoring and modulation with automation system 100 (also called phrenic nerve system 100 herein) can include five components: 1) a set of sensors 140, 2) an input processor 202 that determines which parameters (e.g., the optimal parameters) and the parameter values for the determined parameters to be used for the detection of respiratory activity, 3) a control processor 204 to prescribe the proper actions to be taken, 4) an output processor 206 that determines which parameters (e.g., the optimal parameters) and the parameter values for the determined parameters to be used for the modulation of respiratory activity, and 5) an implantable hardware (e.g., which may include a stimulation electrode and an IPG (Implantable Pulse Generator)) to carry out the sensing, control, and stimulation functions using the parameter values (e.g., that are optimal for such operation) in real time.

FIG. 01 shows a patient 0110 and the phrenic nerve 0120 of the patient 0110. The phrenic nerve system 100 includes a stimulation electrode 0130 that is coupled to the phrenic nerve 0120 of the patient 0110. In certain example embodiments, the stimulation electrode 0130 (which is an example of an implantable device or implantable hardware) can be a cuff type design.

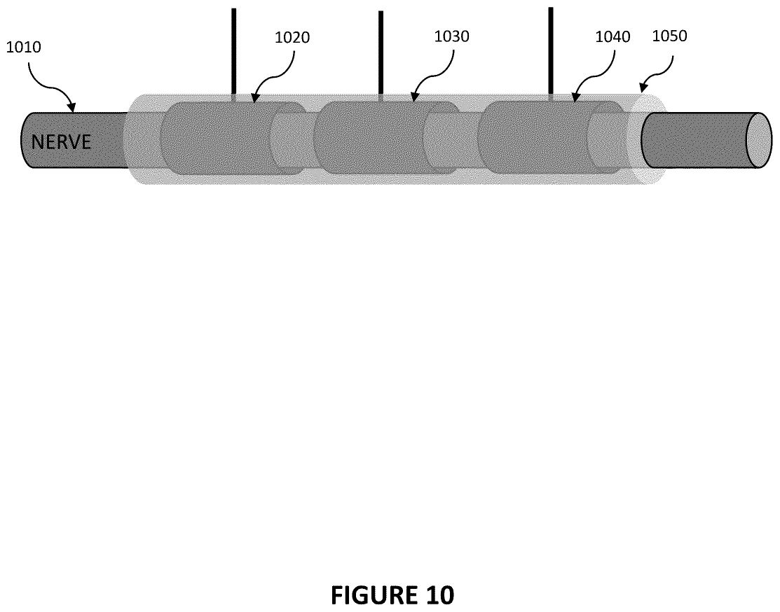

FIG. 10 shows an exemplary implementation of a cuff electrode with three electrodes. Nerve 1010 is in electrical contact with three conductors, 1020, 1030 and 1040. Outer sheet of the cuff electrode, 1050, provides the housing and electrical insulation necessary to prevent the inadvertent stimulation of the non-target tissues around the cuff electrode.

The phrenic nerve system 100 can also include one or more sensors 0140 which are provided to sense physiological characteristics of the patient 0110. Examples of different types of sensors that may be used in certain example embodiments include transthoracic impedance, accelerometer, pleural pressure, oxygen saturation, and the like. Output signals from the sensors (either in digital or analog form) are sent via a sensory signal pathway 0150 to electronics 0170 (discussed below). The sensory signal pathway 0150 may be wired or wireless in order to allow signals from the one or more sensors 0140 to be communicated to another device.

Nerve signal 0160 provides signals regarding sensory nerve firings. Such signals are provided to electronics 0170.

Phrenic nerve system 100 also includes electronics 0170, which can include an input signal processor (also called an input processor 202 as discussed below), control processor 204 (discussed below), output processor 206 (discussed below), and a stimulation circuit. Any or all of the electronics 0170 may be implanted within patient 0110 or may be provided externally to the patient 0110. A stimulation led 0180 is also provided that couples the electronics 0170 to the stimulation electrode 0130.

### Sensors

The set of sensors (e.g., one or more sensors 0140 from FIG. 1) provide the signals going into the input signal processor. Illustrative examples of different types of sensors that may be used in connection with certain example embodiments include any combination of the following: 1) passive electrical sensors that detect the electromyogram (EMG), electrocardiogram (ECG), action potential, compound action potential and electrically evoked compound action potential (eCAP), 2) active electrical sensors such as the transthoracic impedance as well as ultrasound and milli-meter wave radar for motion detection, 3) mechanical sensors such as pressure sensors, accelerometers, gyroscopes, and microphones, and 4) optical sensors such as the tissue oxygen sensors and blood oxygen sensors.

In certain example embodiments, each or any of the sensors produces a signal that is representative of the physical or physiological quantity that is being measured. For example, an acceleration sensor may produce signals that measure acceleration in 1, 2, or 3 axes. The signals that are produced by the sensors may be represented in certain examples as a graph or as waveforms. In certain examples, and as discussed elsewhere herein, the signals from the sensors can be analyzed and one or more values extracted therefrom (e.g., when an acceleration value exceeds a given threshold).

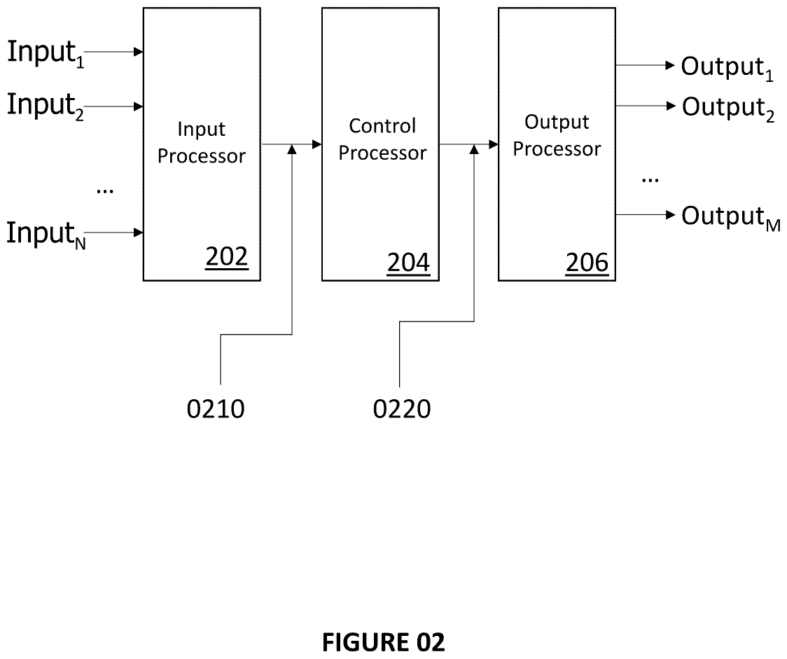

### Description of FIG. 02

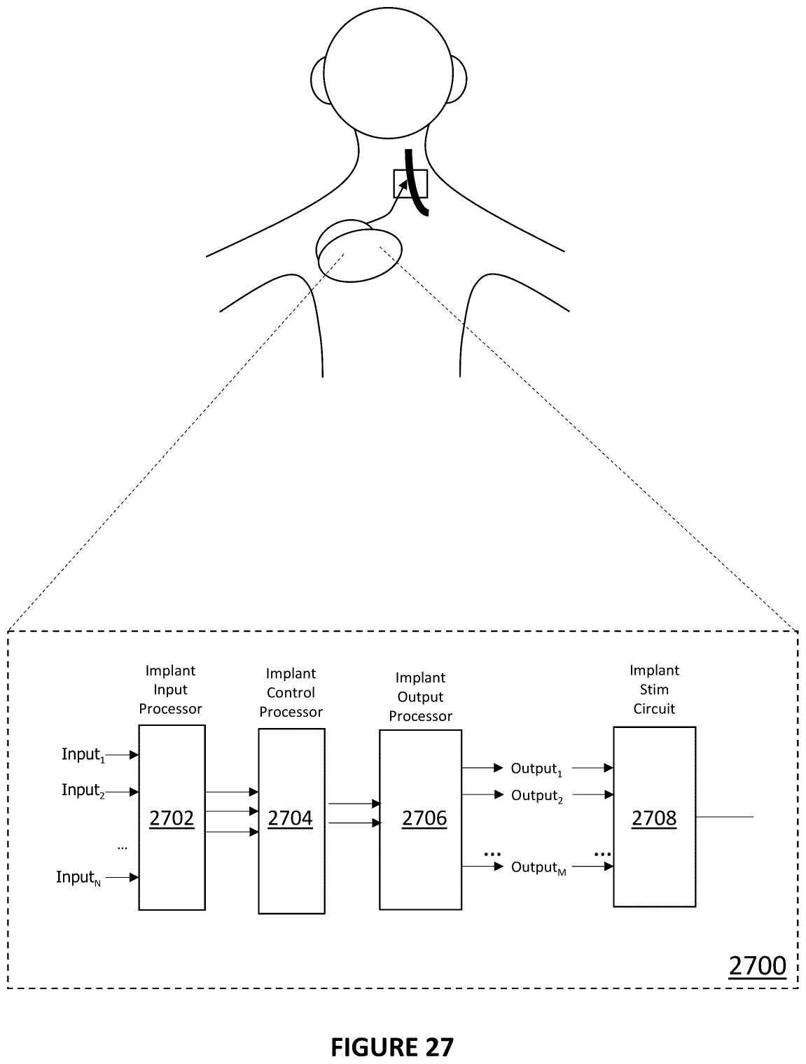

FIG. 02 shows an operational block diagram of components of the phrenic nerve monitoring and modulation system 100. As noted above, the system 100 includes input processor 202, control processor 204, output processor 206. In certain example embodiments (e.g., as discussed in connection with FIG. 27), any or all of the components of the phrenic nerve monitoring and modulation system 100 may be embodied in a device that is implantable into a patient (referred to as an “implantable device” herein). In certain example embodiments, input processor 202, control processor 204, and output processor 206 may be implemented using the same hardware processor and each or any of the functionality provided by the input processor 202, control processor 204, and output processor 206 may be provided in hardware, software, or a combination of hardware and software.

### Input Processor 202

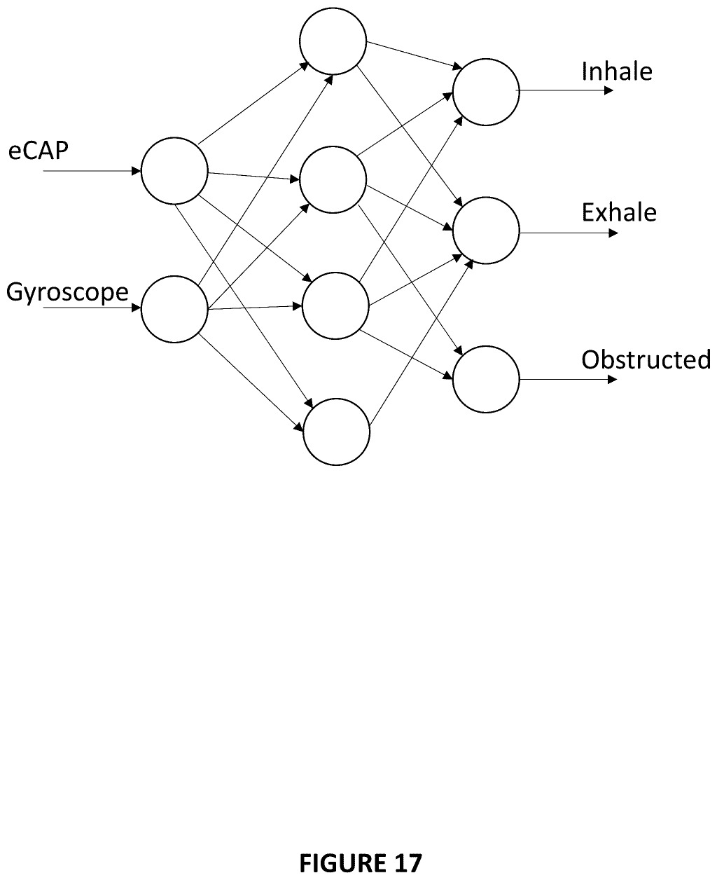

The input processor 202 (also called an input signal processor) is used for processing input signals coming from the sensors (e.g., sensors 0140). Input processor can be included in a computer running an optimization algorithm (referred to as an optimizer herein) that allows the determination of the optimal parameter sets (e.g., determine which features to use, such as whether to use a pressure signal, an accelerometer signal, or both) as well as the parameter values (e.g., which pressure values within a pressure signal are relative thereto) for the detection of the respiratory events. For example, the input processor can be an artificial neural network that is taking the inputs of electrically evoked compound action potentials and gyroscope signals, and producing the respiratory detections, such as inhalation, exhalation, or an obstructed breath, as shown in FIG. 17.

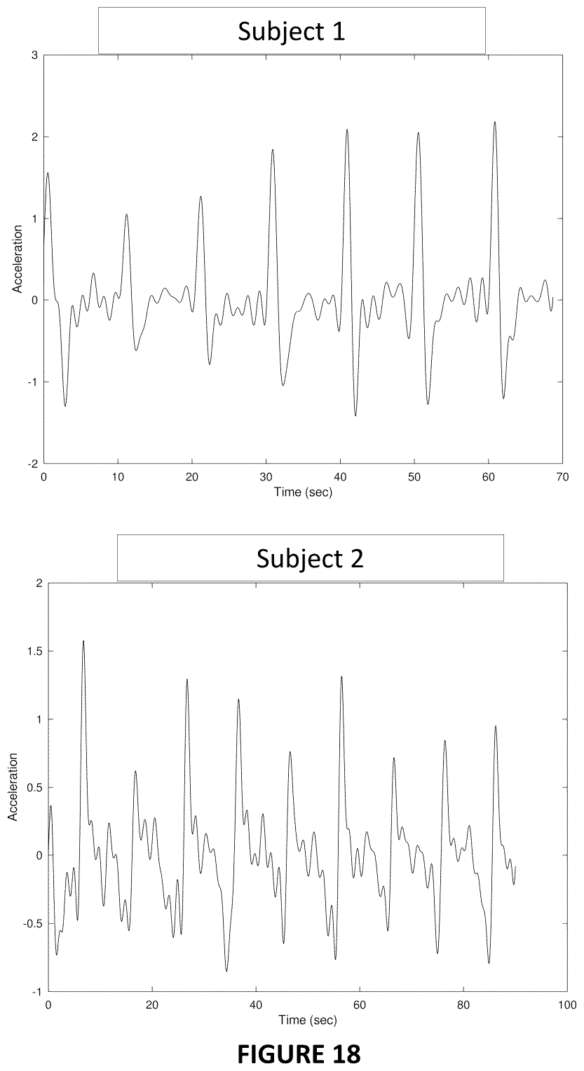

The purpose of the input processor is to determine the optimal set of parameters for the detection of the respiratory events using the available set of inputs. The optimization done by the input processor is necessary as the morphology of the signals coming from different patients for a given outcome can be different. This is illustrated by the gyroscope recordings as shown in FIG. 18. Traces shown in FIG. 18 are the output of the same gyroscope mounted on the same location on the chests of two age and weight matched individuals, lying flat on their back and breathing at a given rate. As it can be seen from FIG. 18, the gyroscope traces obtained from the same location on the chests of the two individuals differ significantly due to the anatomical differences between the two individuals. Similarly, the electrically evoked compound action potentials may also differ from one individual to the next due to their anatomical and physiological differences. Hence, a detector that is residing within the implantable device of a patient may include a set of parameters customized for that patient to detect the respiratory events using the input parameters, and that is what the input signal processor does.

The task of determining the optimum set of parameters can be done in various ways, which depends on the implementation of the input processor. Input processor can be implemented using one or more of algorithms, including, but not limited to: artificial neural network (ANN) or fuzzy logic, convolutional neural network, generative adversarial network (GAN), auto-encoder, Bayesian optimizer, maximum likelihood estimator, linear support vector machine, and/or classification and regression trees (CART).

In certain examples, the input processor may produce outputs in ON/OFF format or in percent certainty.

In some embodiments, the optimizer is based on an artificial neural network, or a fuzzy logic, and the optimization is achieved using the back propagation of error method. Below is pseudo code for training an artificial neural network (ANN) with m sets of input data input, where each data set contains n inputs.

```
TABLE 1
Step 01: Define X ← Training Data Set of size mxn
Step 02: Define y ← Outputs for records in X
Step 03: Define ω ← Initial guesses for the weights for respective layers
Step 04: Define l ← The number of layers in the neural network, 1,..,L
Step 05: Define a^{(l)} ← outputs of the neurons in the neural network
Step 06: Define b^{(l)} ← bias values for the neurons in the neural network
Step 07: Propagate forward: z^{(l )}ⱼ ← Σ ω^{(l-1)}ⱼₖ a^{(l-1)}ₖ + b^{(l-1)}
    a^{(l )}ⱼ ← σ[ z^{(l )}ⱼ ]
Step 08: Calculate cost Function: C ← (1/N_{L}) Σ (a^{(l)} − y)²
Step 09: if C is small, then halt, else continue
Step 10: Calculate partial derivatives: ∂C / ∂ω^{(l-1)}ⱼₖ & ∂C / ∂b^{(l-1)}
Step 11: Update neural network coefficients:
  ω^{(l-1)} (t+1) ← ω^{(l-1)} (t) − η ∂C / ∂ω^{(l-1)}
  b^{(l-1)} (t+1) ← b^{(l-1)} (t) − η ∂C / ∂b^{(l-1)}
Step 12: Go to Step 07
```

Ultimately, the coefficients of the neural network ANN-1 are passed (e.g., wirelessly communicated, etc.) to the implantable device to be used during runtime.

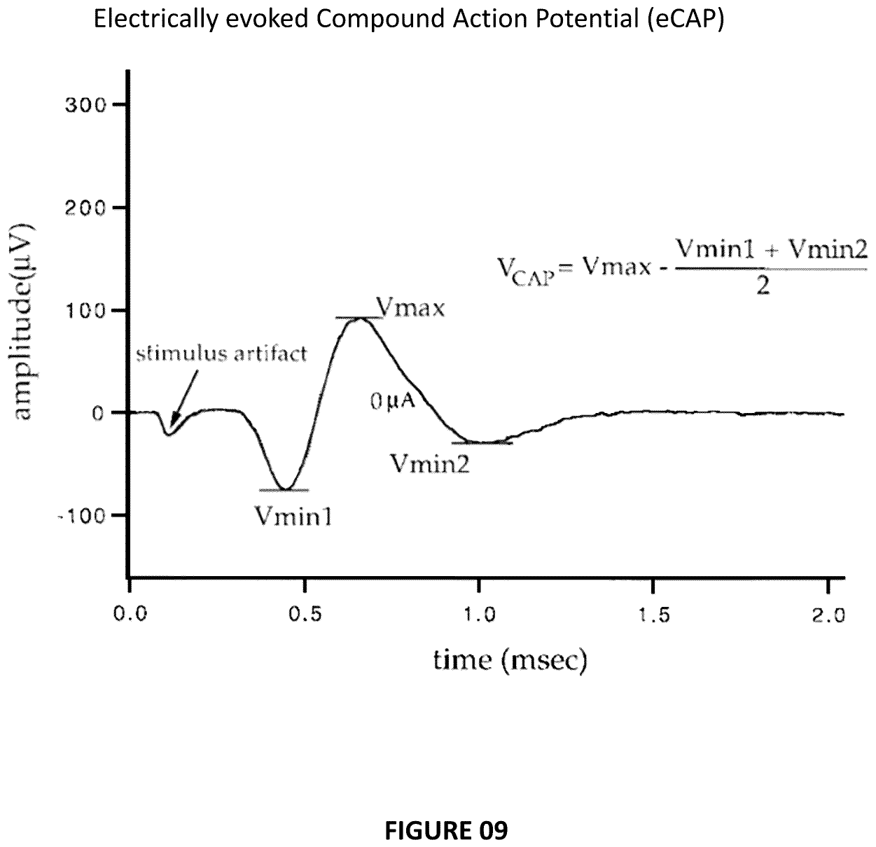

In some embodiments, the optimizer is based on a convolutional neural network (CNN) which is a modified neural network that has at least one convolution layer that is used for obtaining localized information. For example, instead of or in addition to the actual signal of electrically evoked Compound Action Potential (eCAP), a feature extracted from the eCAP, such as VCAP can be fed into the next layer of the neural network. As illustrated in FIG. 9, VCAP can be calculated using the following formula:

$${V}_{CAP} = {V}_{MAX} - ( {V}_{MIN 1} + {V}_{MIN 2} ) / 2 \qquad Equation 01$$

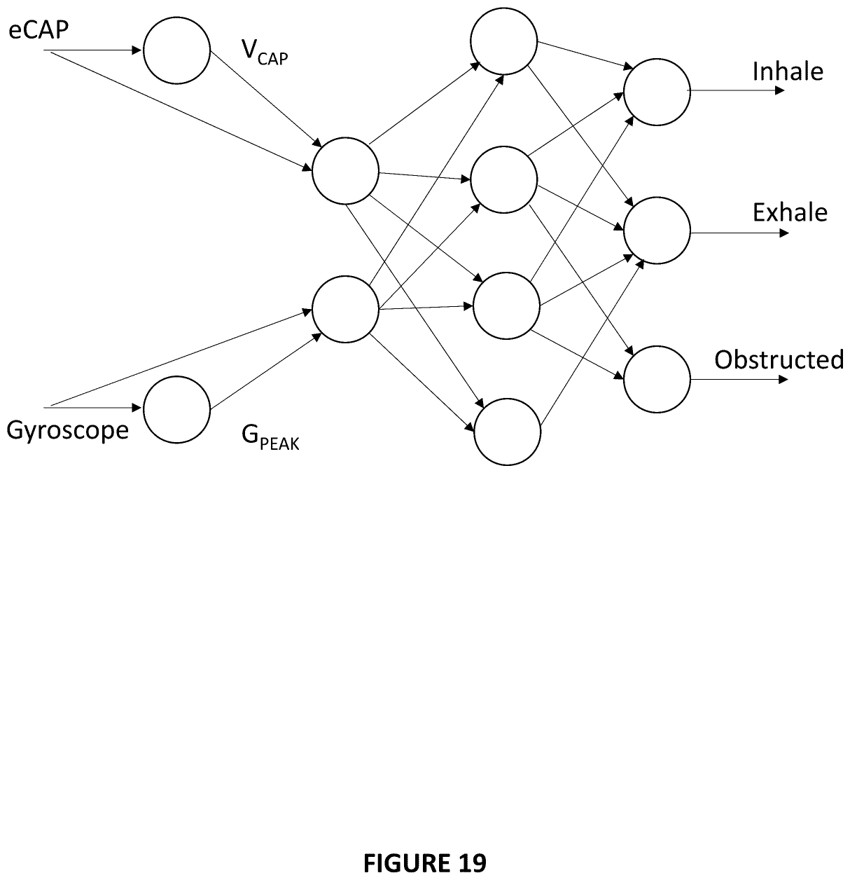

FIG. 19 illustrates an exemplary implementation of a convolutional neural network where the convolution layer extracts the features such as VCAP from eCAP and G_{PEAK} from gyroscope signals. These are then provided as additional inputs to the neural network as is shown in FIG. 19. V_{CAP} is obtained from eCAP using the Equation 01 and G_{PEAK} derived from the gyroscope signals using a peak detector. The use of features, such as V_{CAP} and G_{PEAK} that were extracted from the eCAP and gyroscope signals, can be used to provide a richer set of inputs to the layers of the artificial neural network. This in turn can improve the ability of the NN to correctly identify (e.g., the accuracy) the desired respiratory events (e.g., inhale, exhale, obstruction, etc.).

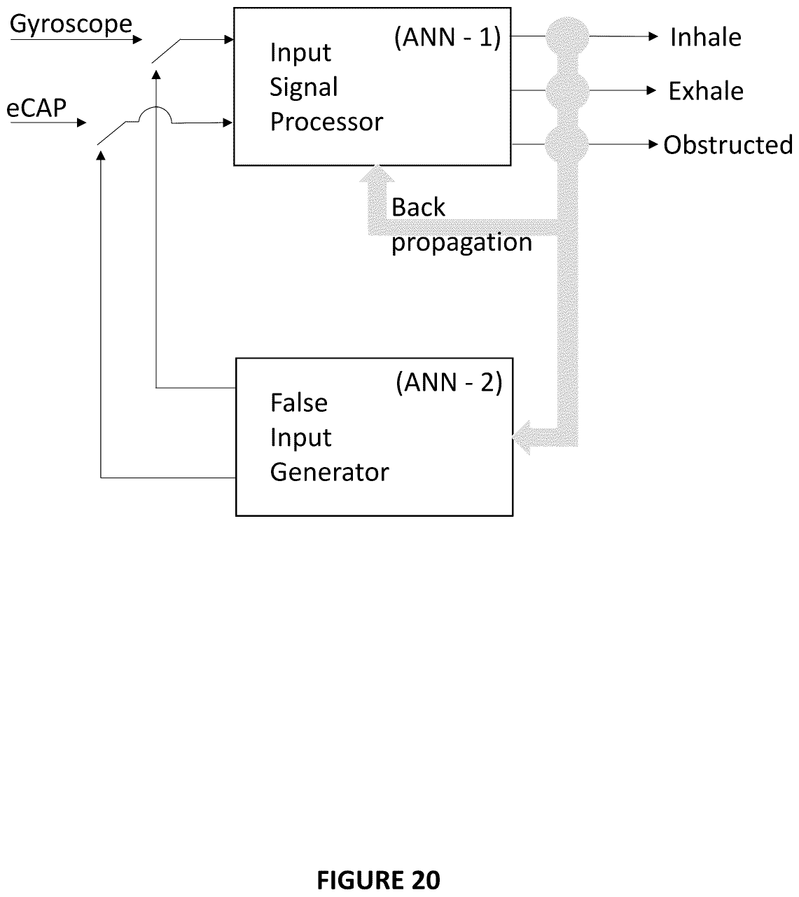

In some embodiments, the optimizer is based on a generative adversarial network (GAN), as illustrated in FIG. 20. In this scenario, two neural networks, ANN-1 and ANN-2 work against each other. While ANN-1, which is the Input Signal Processor, works to correctly detect the respiratory events, such as inhalation, exhalation, and obstructions using actual eCAP signals, ANN-2 works to generate false signals in an attempt to fool ANN-1. With the help of ANN-2, ANN-1 learns and becomes a better distinguisher of various respiratory events. Implementation of the scheme is similar to the simple artificial neural network where the cost function is given as follows:

$$Cost = ( 1 / {N}_{L} ) \sum {( {a}^{( l )} - y )}^{2} \qquad Equation 2$$

In the GAN implementation, the Input Signal Processor aims to minimize the cost function while the false input generator aims to maximize the cost function. Ultimately, the coefficients of the neural network ANN-1 are passed to the implantable device to be used during runtime.

In some embodiments, the optimizer is based on the autoencoder scheme where the Input Signal Processor learns to capture the most important parts of the input signal, which is important for the processing of noisy signals. Autoencoder works by first severely compressing the data, and then expanding it, using the three groups of layers of a neural network which are labelled encoder, bottleneck, and decoder.

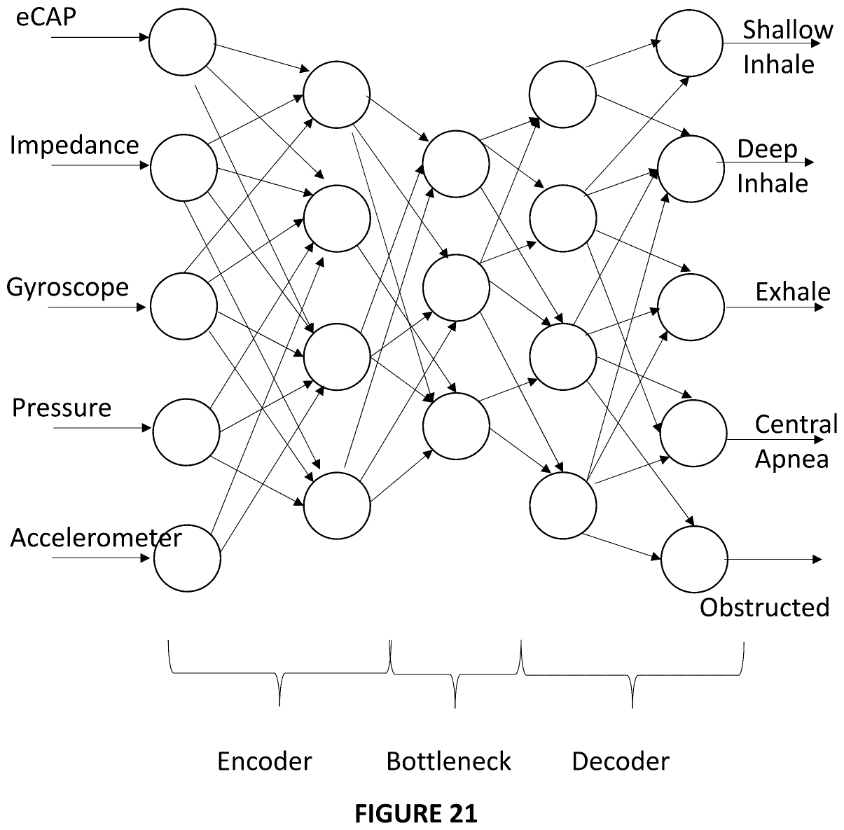

As shown in FIG. 21, few of the input layers of the neural network are used as an encoder where the number of neurons in each successive layer is reduced—until reaching the bottleneck. After passing through the bottleneck, the number of neurons increase in each layer until getting to the output layer. The compression process of the encoder forces the autoencoder to learn the important features of the input signals while ignoring the noise that is present on the input signal. It should be noted that even though the inputs to the autoencoder shown in FIG. 21 are represented as the raw signals coming from the sensors, it is also possible to send the features such as V_{CAP} and G_{PEAK} that were extracted from the eCAP and gyroscope signals as inputs to the autoencoder. Ultimately, the coefficients of the neural network forming the autoencoder are passed to the implantable device to be used during runtime.

In some embodiments, the optimizer is based on the Bayesian optimizer scheme where optimization is done in a sequential manner and does not require an a priori model. Instead, the Bayesian optimizer first constructs a surrogate function, and then uses it to optimize the objective function while applying Bayesian machine learning techniques to reduce the uncertainty in the surrogate function. Below is pseudo code that can be used for an implementation of a Bayesian optimizer according to certain example embodiments, starting with the warm up phase followed by the post warm up phase:
//Bayesian optimization for warm up phase:
for i=1 to N_{WARMUP} do
   Record input signal: xᵢ←[eCAPᵢ, gyroscopeᵢ]
   Record output signal: yᵢ←[inhaleᵢ, exhaleᵢ, obstructedᵢ]
end for //end of the warmup phase

Following the warmup phase, a surrogate function representing the relationship between the input and output is constructed which is given as:

$${y}_{i} = f ( {x}_{i} ) \qquad Equation 3$$

During the post warm up phase, the model fitness is evaluated, and the surrogate model is updated to minimize the cost function and to minimize the uncertainties in the model.

$$Cost = \sum {[ f ( {x}_{i} ) - {y}_{i} ]}^{2} \qquad Equation 4$$

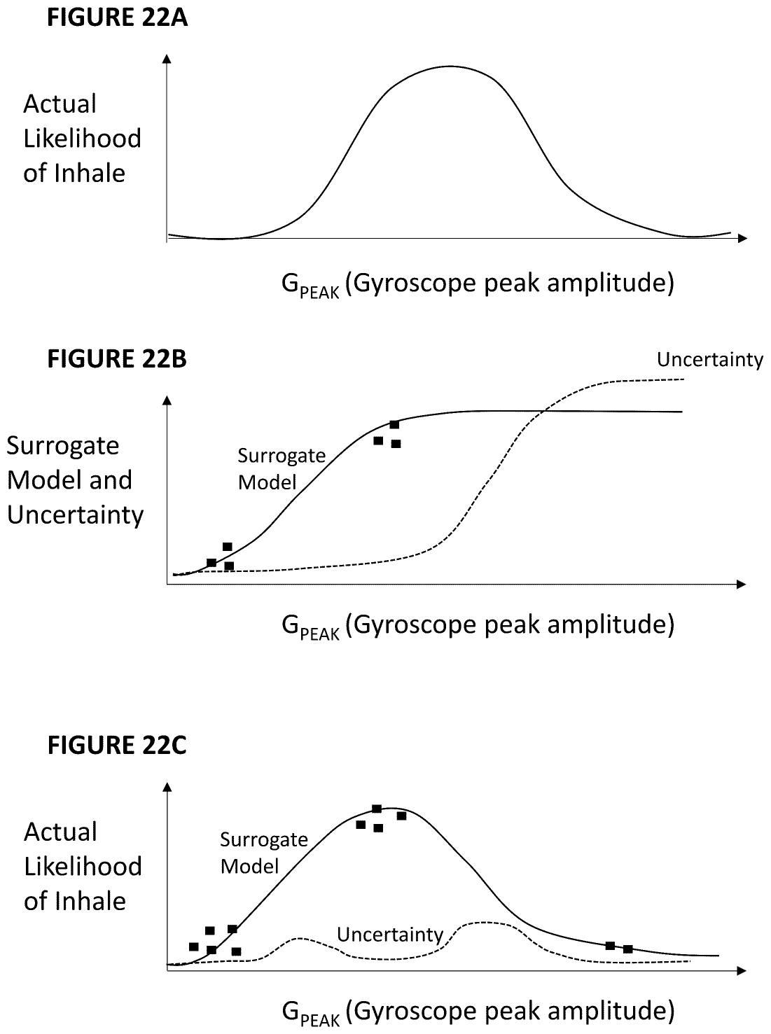

Reduction in the uncertainties of the surrogate model may determine the length of the post warm up phase. This is further illustrated in FIGS. 22A-22C. FIG. 22A shows an example where the actual likelihood of inhalation is given as a function of the input variable G_{PEAK}, which is defined as the peak value of the gyroscope input. Unfortunately, this relationship between G_{PEAK} and Inhale is not known to the system, and it must be learned without any a priori knowledge, which is what is done by the Bayesian optimizer. After some data becomes available, the algorithm goes thru the warmup phase, and learns the behavior to construct the surrogate model as shown in FIG. 22B. It should be noted that the black squares in FIG. 22B represent the data points and the solid line represents the surrogate model. Since there was no data at certain regions of the input domain, such as the instances with high values of G_{PEAK}, the uncertainty in those regions remains high. During the post warm up phase, the algorithm continues to learn and updates the surrogate model. The post warm up phase ends when the uncertainty across the entire domain falls below a given threshold, as illustrated in FIG. 22C.

Above-described implementation of the Bayesian optimizer differs from the traditional implementations of the Bayesian optimizers used in artificial intelligence systems as it does not utilize an acquisition function (e.g., a selection function) to determine the next input parameter at any given time. Instead, the system uses whatever signal is produced by the patient during the post warm up phase.

A surrogate function for a Bayesian optimizer can be chosen by any method that is preferred. In certain examples, function can be selected so that the distribution in the input data is assumed to be Gaussian in nature and surrogate model conforms to it. Ultimately, the resulting surrogate function is passed to the implantable device to be used during the runtime.

In some embodiments, the optimizer is based on a maximum likelihood estimator. In this case, the input signal processor receives the inputs and the outputs for training and records it. As it is done in Bayesian optimizer:
Record input signal: xᵢ←[V_{CAP_i}, G_{PEAK_i}]
Record output signal: yᵢ←[inhaleᵢ, exhaleᵢ, obstructedᵢ]

Afterwards, the mean and the standard deviation of each input variable is calculated, and also for each output condition. For example, if there were N_{INH} samples for inhale, N_{EXH} samples for exhale and N_{OBS} for obstructed breaths, then the following statistics will be calculated:
μ_{VCAP_INH=1}=mean of V_{CAP} values for inhales;
σ_{VCAP_INH=1}=standard deviation of V_{CAP} values for inhales;
μ_{VCAP_INH=0}=mean of V_{CAP} values for exhales and obstructed breaths;
σ_{VCAP_INH=0}=standard deviation of V_{CAP} values for exhales and obstructed breaths;
μ_{GPEAK_INH=1}=mean of G_{PEAK} values for inhales;
σ_{GPEAK_INH=1}=standard deviation of G_{PEAK} values for inhales;
μ_{GPEAK_INH=0}=mean of G_{PEAK} values for exhales and obstructed breaths; and
σ_{GPEAK_INH=0}=standard deviation of G_{PEAK} values for exhales and obstructed breaths.

Similar mean and standard deviation values of V_{CAP} and G_{PEAK} are calculated for both exhale only and obstructed breath only. Once mean and standard deviation values are calculated, the learning phase for the maximum likelihood estimator is completed. When a new data set of xₙ←[V_{CAP_n}, G_{PEAK_n}] is received, the predicted value of each output, e.g., Probability of Inhaleₙ, Probability of Exhaleₙ, and Probability of Obstructedₙ can be determined using the predetermined distribution functions:

Probability of Inhaleₙ= [Probability of Inhaleₙ given V_{CAP_n}]×[Probability of Inhaleₙ given G_{PEAK_n}] or

$$P ( {Inhale}_{n} ) = P ( {Inhale}_{n} \vert {V}_{{CAP}_{n}} ) \cdot P ( {Inhale}_{n} \vert {G}_{{PEAK}_{n}} ) \qquad Equation 5$$

where the probabilities are calculated using the probability density function given as follows:

Probability of Inhaleₙ given V_{CAP_n}

EQUATION 6
$P ( {Inhale}_{n} {}_{\vert} {V}_{{CAP}_{n}} ) = \frac{1}{[ {σ}_{{V}_{CAP INH} = 1} ] \cdot \sqrt{2 π}} {e}^{- {\frac{1}{2} [ \frac{{V}_{{CAP}_{n}} - {μ}_{{V}_{CAP INH} = 1}}{{σ}_{{V}_{CAP INH} = 1}} ]}^{2}}$

and Probability of Inhaleₙ given G_{PEAK_n}

EQUATION 7
$P ( {Inhale}_{n} {}_{\vert} {G}_{{PEAK}_{n}} ) = \frac{1}{[ {σ}_{{G}_{PEAK INH} = 1} ] \cdot \sqrt{2 π}} {e}^{- {\frac{1}{2} [ \frac{{G}_{{PEAK}_{n}} - {μ}_{{G}_{PEAK} INH = 1}}{{σ}_{{G}_{PEAK INH} = 1}} ]}^{2}}$

Similarly, the Probability of Exhaleₙ as well as the Probability of Obstructed breathₙ can be calculated using the corresponding mean and standard deviation values of the V_{CAP} and G_{PEAK}.

Ultimately, all mean and standard deviation values calculated above are downloaded into the implantable device to be used during the runtime.

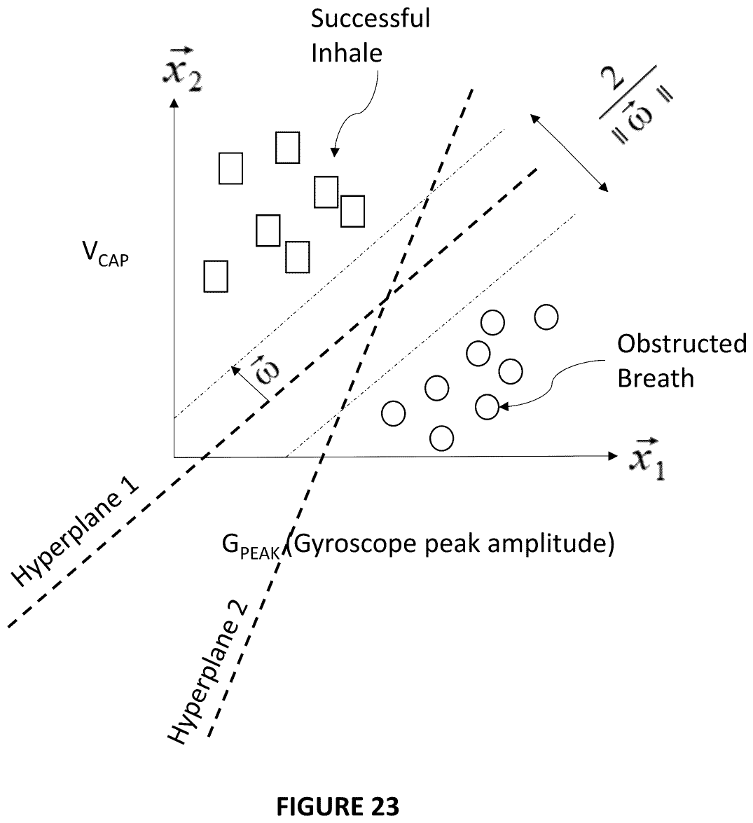

In some embodiments, the optimizer is based on a linear support vector machine. In this case, the input processor 202 receives the inputs and the outputs for training, and forms a classifier, as illustrated in FIG. 23. Once again, the example illustrates a case where only two inputs are used by the input signal processor, namely the gyroscope and eCAP signal. Furthermore, example assumes that the features V_{CAP} and G_{PEAK} are extracted from the gyroscope and eCAP signals respectively. In FIG. 23, the inputs to the input processor are labelled as x₁ and x₂.

In FIG. 23, classification of the inputs using the input variables x₁ and x₂ are illustrated by grouping the breaths resulting in successful inhales and obstructed breaths. Illustration indicates the successful breaths as rectangular shapes and the obstructed breaths as round circles. Such a data set can be obtained during the training phase and used for learning the algorithm to distinguish future breaths as they are experienced by the implantable device later on.

Demarcation line for different outputs (y) in FIG. 23 is shown as the hyperplane separating the two sets of outputs, e.g., successful inhales and obstructed breaths. Even though both hyperplane 1 and hyperplane 2 would separate the outputs successfully, hyperplane 1 is preferred over hyperplane 2 as it provides a more consistent separation between the two groups of outputs, as indicated by the direction of ω. Hence, the goal of the optimizer during the learning phase is to come up with the hyperplane that would generate the maximum separation between the two groups of outputs, e.g., successful inhale and obstructed breaths, and with highest consistency. In other words, the optimizer works to minimize ω, or maximizes:

$$\frac{2}{‖ \vec{ω} ‖}$$

Operation of the optimizer for the input signal processor using the linear support vector machine is described below. Input to the optimizer is defined as a vector:

\vec{x}ᵢ=[x₁_{ᵢ},x₂_{ᵢ},]
where i is the index for the input, which is formed by the two inputs, such as V_{CAP} and G_{PEAK} in this example. Each output from the input signal processor would be labeled as yᵢ and would be given as:

EQUATION 8
${y}_{i} = \{ \begin{matrix}+ 1 , if successful inhale \\ - 1 , if obstructed breath\end{matrix}$

Requirement for the optimization would be:

$$\vec{ω} \cdot {\vec{x}}_{i} - b \geq 1 if {y}_{i} = + 1$$

$$And$$

$$\vec{ω} \cdot {\vec{x}}_{i} - b \leq 1 if {y}_{i} = - 1$$

or the general requirement of:

$${y}_{i} ( \vec{ω} \cdot {\vec{x}}_{i} - b ) \geq 1 for all 1 \leq i \leq N$$

where N is the total number of samples in the training set. Therefore, the overall optimization requirement would be to:

$$minimize ‖ ω ‖ while {y}_{i} ( \vec{ω} \cdot {\vec{x}}_{i} - b ) \geq 1$$

Above optimization problem is a non-linear constrained optimization, and can be solved using any known method, such as AMEOBA and relaxation method.

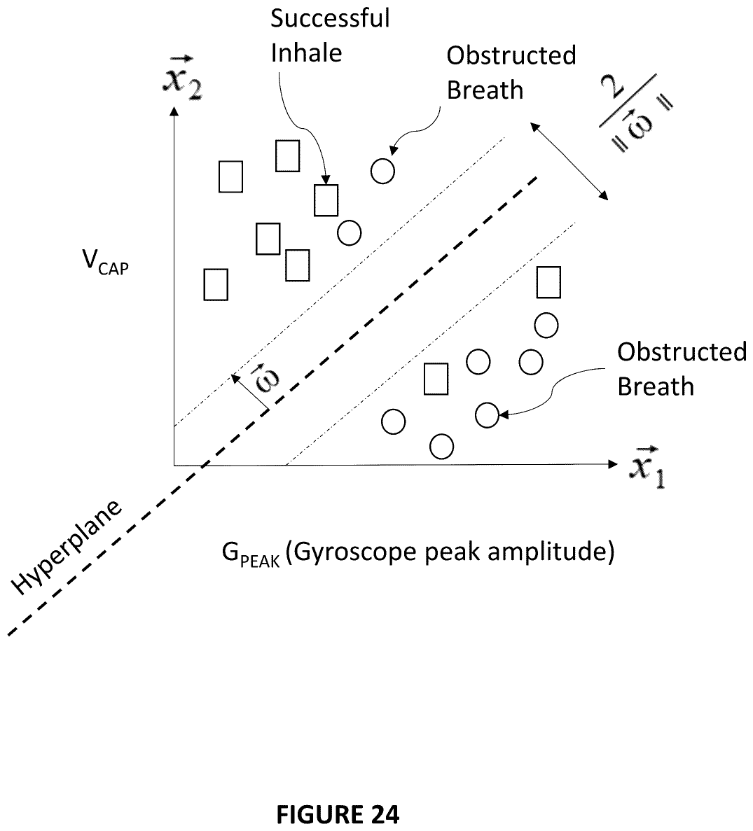

FIG. 24 illustrates a case where the complete separation of the outcomes may not be possible. In that case, the linear support vector machine implements a hinge loss function which is given as:

$$maximum \{ 0 , {y}_{i} ( \vec{ω} \cdot {\vec{x}}_{i} - b ) \}$$

$$where$$

$${y}_{i} ( \vec{ω} \cdot {\vec{x}}_{i} - b ) = \{ \begin{matrix}+ {y}_{i} , & if classified correctly \\ - {y}_{i} , & if classified incorrectly\end{matrix}$$

$$and$$

$$1 - {y}_{i} ( \vec{ω} \cdot {\vec{x}}_{i} - b ) = \{ \begin{matrix}0 , & if classified correctly \\ 2 , & if classified incorrectly\end{matrix}$$

Then, the goal of the optimizer would be to:

$$minimize [ \frac{1}{N} {\sum }_{i = 1}^{N} maximum \{ 0 , {y}_{i} ( \vec{ω} \cdot {\vec{x}}_{i} - b ) \} + λ \cdot {‖ \vec{ω} ‖}^{2} ]$$

where the second term above represents the Lagrange multiplier for the constraint. Again, the above minimization is a multi-dimensional, non-linear optimization problem which can be solved numerically using any one of the algorithms, such as AMEOBA or relaxation.

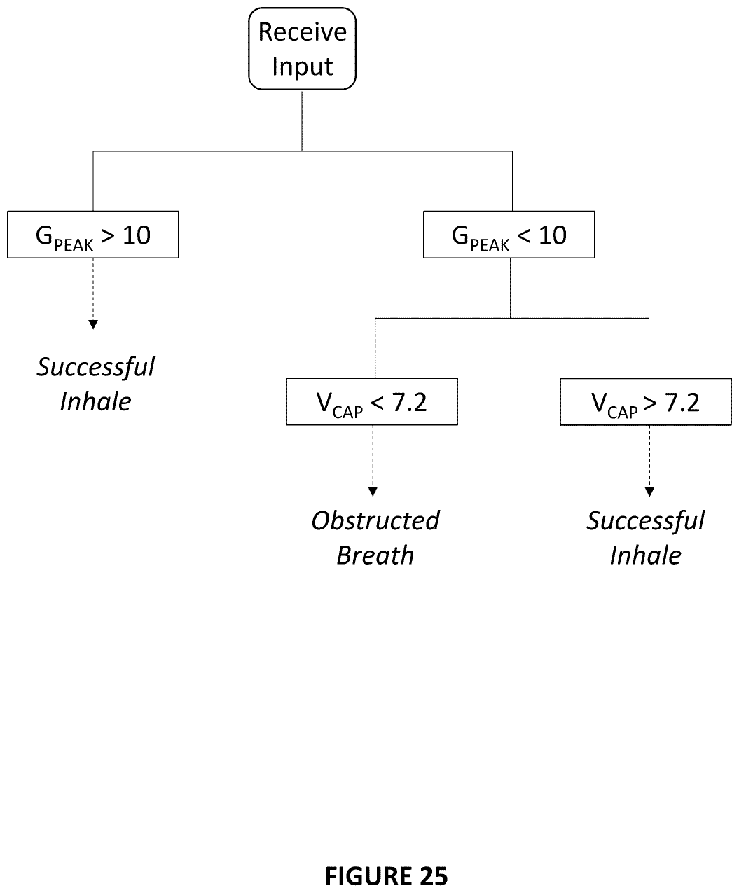

In some embodiments, the optimizer is based on a classification and regression trees (CART). In this case, the input processor 202 receives inputs (e.g., G_{PEAK} and V_{CAP}) and then produces the outputs, such as the obstructed breath or normal inhalation. Input processor that is implemented using the CART algorithm accomplishes this task by classifying the input patterns into categories, such as obstructed breath or normal inhalation, which is done based on cut offs or limits put on the parameters, as illustrated in an examples shown in FIG. 25. Once these cut offs, such as the value of 10 for G_{PEAK}>10, are determined, cut offs as well as the overall tree structure are downloaded into the implantable device for use during the runtime. The example classification algorithm shown in FIG. 25 uses two inputs, but it will be appreciated that one, or three or more inputs can be used in accordance with certain example embodiments. Furthermore, the tree can be deeper or shallower than the one that is shown in FIG. 25. The task of the optimizer is to determine the cut-off values (e.g., parameter values), such as the 10 for G_{PEAK} and 7.2 for V_{CAP} as shown FIG. 25, using a process as described below.

In certain example embodiments, and objective of the optimizer using CART is to minimize the following objective function:

$$minimize \{ \frac{1}{N} {\sum }_{i = 1}^{N} {[ {y}_{i} - {prediction}_{i} ]}^{2} \}$$

To find the cut-offs, aka splitting points, the CART optimizer defines a score called the Gini index, which is an indication of the purity of the samples that are within a given selection box:
Gini=0.00→pure
Gini=0.50→50-50 split, and so on

For a binary classification problem, such as successful inhale versus obstructed breath, there will be two probabilities or proportions in each box, P₁ and P₂. P₁ and P₂ represent the proportions of successful inhales and obstructed breaths in each box respectively, and their sum must be equal to one.

$${P}_{1} + {P}_{2} = 1 , or \qquad [ Equation 09 ]$$

$${( {P}_{1} + {P}_{2} )}^{2} = 1 \qquad [ Equation 10 ]$$

Rewriting:

$${{P}_{1}}^{2} + 2 {P}_{1} {P}_{2} + {{P}_{2}}^{2} = 1 \qquad [ Equation 11 ]$$

$$or$$

$$2 {P}_{1} {P}_{2} = 1 - ( {{P}_{1}}^{2} + {{P}_{2}}^{2} ) = Gini \qquad [ Equation 12 ]$$

Therefore, the Gini index for a chosen split point in a binary classification problem will be:

EQUATION 13
$Gini = [ 1 - ( {g}_{11}^{2} + {g}_{12}^{2} ) ] \frac{{n}_{{g}_{1}}}{n} + [ 1 - ( {g}_{21}^{2} + {g}_{22}^{2} ) ] \frac{{n}_{{g}_{2}}}{n}$

where
   g_{i,j} is the proportion of instances in group i for class j,
   n_{g1} is the total number of instances in group 1,
   n_{g2} is the total number of instances in group 2, and
   n is the total number of instances from the parent box.

In certain example embodiments, an optimization process is carried out to reduce all the Gini scores. This can be performed by, for example, an exhaustive search, steepest descent, or any other optimization scheme. Afterwards, the resulting CART structure along with the cut-offs are downloaded into the implantable device for run time use.

It should be noted that regardless of the implementation of the optimizer, such as artificial neural network (ANN), convolutional neural network, generative adversarial network (GAN), auto-encoder, Bayesian optimizer, maximum likelihood estimator, linear support vector machine, or a classification and regression trees (CART), an objective of the input processor is to learn to interpret the patient specific signals from the sensors and determine the optimal algorithm to do so. In some embodiments, the type of algorithm that is downloaded into an implantable device can be determined or otherwise set manually (or automatically). As an illustrative example, the type of algorithm may be set to be an ANN. In such a case, the training phase may include the determination of the weights of the ANN. In other embodiments, more than one algorithm can be tried. For example, an ANN, GAN, and CART can be deployed and subsequently trained. When multiple types of algorithms are used, then there may be another selection process to determine which one of the algorithms to use. For example, the algorithm that produces the most consistent classification during the training phase can be selected as the winning algorithm. The winner (along with any parameters or other characteristics that have been determined as part of the training phase) can be provided to an implantable device. In certain example embodiments, a winning algorithm can be selected using Equation 14 below. As shown below the total error is the sum of all individual prediction errors, Errorᵢ. For example, an individual prediction error can be defined as 1 if the actual event and the predicted event are not the same, and 0 if the actual event and the predicted event are the same.

$$TotalError = \sum [ {Error}_{i} ] \qquad Equation 14$$

The winning algorithm that is then selected can be used by the implantable device as the input processor 202 which provides improved (e.g., maximum) accuracy.

### Control Processor 204

Control processor 204 receives the outputs produced by the input processor 202 and generates at least one action that is to be taken. Inputs to the control processor 204 from the input processor can be the interpretation of the status of the respiratory function of the patient. Parameters provided by the input signal processor to the control processor 204 may include, but are not limited to: arousal, inhalation, exhalation, obstructed breath (obstructed apnea), heart rate, hypopnea, and apnea-hypopnea index.

Furthermore, the above listed parameters may be in the form Boolean variables (e.g., zero or one), or in a continuous form, such as a probability in the form of a real number in the range of 0 to 1.

Control processor utilizes the information that is gathered by the input signal processor to generate the commands to be passed to the output processor to maintain a minimum respiratory rate. The commands sent by the control processor to the output processor may include: 1) stimulate efferents, 2) stimulate afferents; 3) set duration (with an indicated duration); 4) turn on; and 5) turn off.

### Output Processor 206

Output processor 206 is trained to generate the features of the electrical stimulation waveforms to modulate the physiological system based on the commands it receives from the control processor. Output processor 206 generates one or more outputs. Examples of outputs that may be generated in certain example embodiments include: stimulation amplitude, stimulation pulse width, stimulation frequency, number of pulses in a train, and train timing.

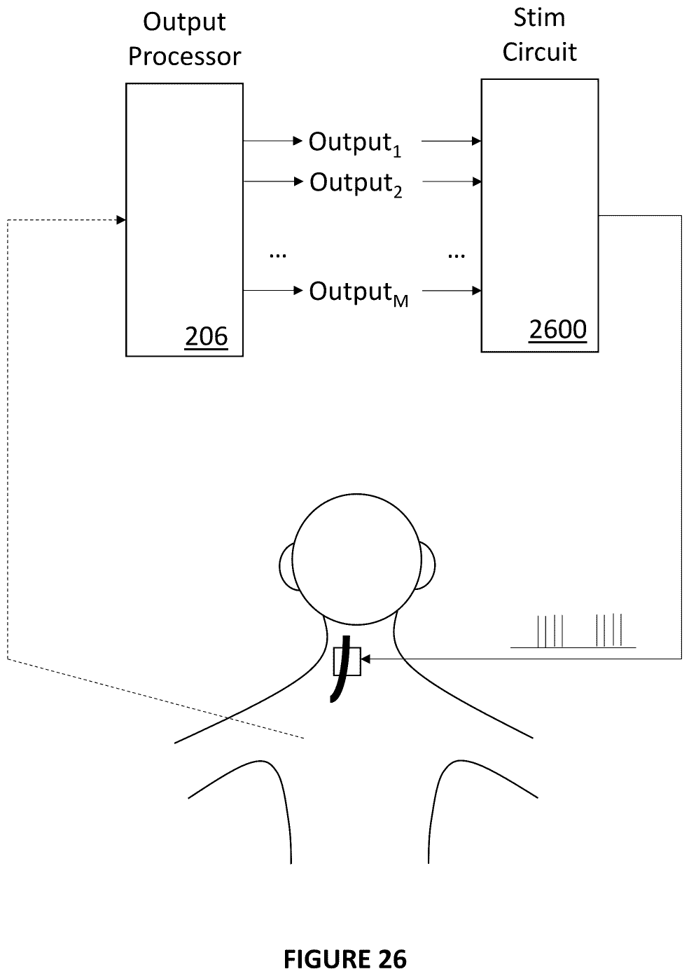

During the training phase, the output processor 206 generates the outputs that are used by the stimulation circuit (an example of which may be a stimulation electrode 0130 as shown in FIG. 1) to generate the electrical pulses to be delivered to the phrenic nerve. Resulting respiratory outcomes are fed back into the output processor 206 to train it to produce the outputs that are required to stimulate the given patient, as illustrated in FIG. 26. As in the case of the input processor 202, output processor 206 can also use any one or more of the following to generate its outputs from the commands that it receives from the control processor: artificial neural network (ANN) or fuzzy logic, convolutional neural network, generative adversarial network (GAN), auto-encoder, Bayesian optimizer, maximum likelihood estimator, linear support vector machine, and classification and regression trees (CART).

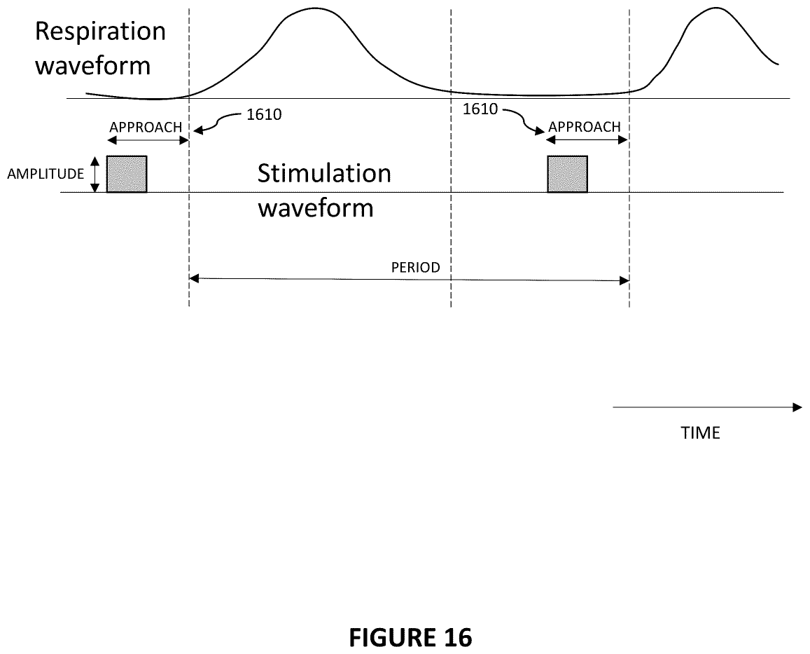

FIG. 16 shows an exemplary timing diagram of the stimulation pattern in reference to the respiration waveform representing the tidal volume of the patient. In this example, the stimulation is delivered in such a way that the onset of the burst train occurs before the onset of the inhalation. The time interval between the onset of the stimulation and the onset of the inhalation is defined as “APPROACH” 1610.

Implementation of the optimization algorithms for the output processor 206 may be similar to those that were described for the input processor 202. However, the inputs to the output processor may be the commands from the control processor 204 and the outputs from the output processor are the stimulation parameters that are provided to the stimulation circuitry (e.g., stimulation amplitude, stimulation pulse width, stimulation frequency, number of pulses in a train, approach duration, and/or train timing).

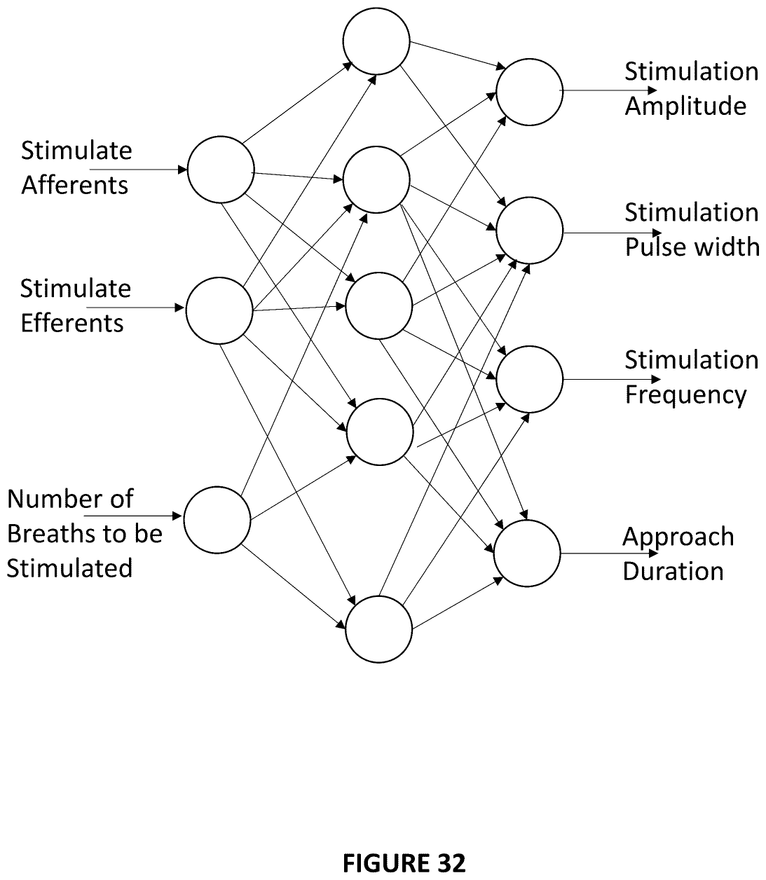

An artificial neural network (ANN) implementation for the output processor is illustrated in FIG. 32. Inputs to the ANN output processor can be the commands that are being received from the Control Processor, such as “Stimulate Afferents”, “Stimulate Efferents” and “Number of Breaths to be Stimulated” (or values that represent those commands). It will be appreciated that although the illustration provided in FIG. 32 shows a single hidden layer, additional hidden layers may be included in the ANN according to certain example embodiments.

Outputs of the control processor can be stimulation amplitude, stimulation pulse width, stimulation frequency and the approach duration. These are then fed into the ANN for processing. Based on the command inputs that the Output Processor receives, an ANN produces the outputs which are converted into the stimulation pulses, as shown in FIG. 26.

During a training phase, the outcomes following the delivery of the stimulation may be evaluated, and the outcomes are categorized. For example, if the stimulation causes a contraction of the diaphragm, then the outcome is the stimulation of the efferents. If the input command was “Stimulate Efferents”, then the desired outcome is achieved. If the input command to the Output Processor was “Stimulate Efferents,” but there is no excitation of the diaphragm, then efferent stimulation was not achieved. This information is used for the updating of the weights of the ANN using the algorithm that is listed in Table 01. Overall, the Control Processor provides the command inputs to the Output processor, and the Output processor produces one or more Stimulation Parameters (e.g., amplitude, pulse width, etc.)

With these determined parameters, the stimulation Circuit generates the output pulses and delivers them to the patient, and the outcomes, such as successful stimulation of efferents, resulting in the contractions of the target muscle, such as the diaphragm, or the lack of contractions of the diaphragm are used as the feedback for the updating of the weights.

Similarly, the outcome of input command “Stimulate Afferents” is evaluated as improved airflow resulting from the improved tone of the upper airway muscles. If the input command to the Output Processor was “Stimulate Afferents”, but there is no improvement in the airflow, then the afferent stimulation was not achieved. This information is again used for the updating of the weights of the ANN using the algorithm that is listed in Table 01.

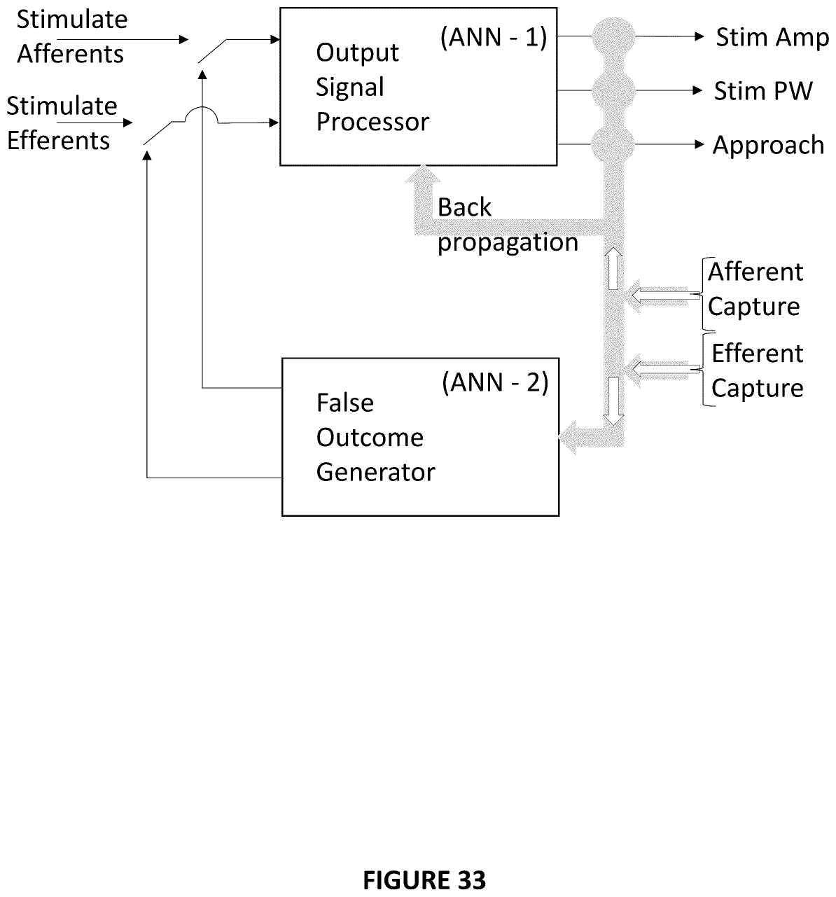

FIG. 33 shows the generative adversarial network (GAN) type implementation that may be used by the output processor according to certain example embodiments. In certain example embodiments (e.g., similar to the input processor), the output processor may include a GAN using two ANNs, where ANN-1 works in the forward direction to generate desired stimulation parameters, such as the stimulation amplitude, pulse width, and frequency for given commands, such as “Stimulate Afferents” and “Stimulate Efferents”. ANN-2 meanwhile can produce random or predetermined outcomes, such as “failed stimulation of efferents” for further fine tuning of ANN-1.

For the stimulation of efferents, excitation may need to be delivered to excite the phrenic nerve and to cause the subsequent contraction of the diaphragm. For the stimulation of the afferents to cause the opening of the airway in advance of the inhalation, stimulation may need to be delivered in advance of the onset of inhalation, which is the time period defined as “approach”, and the stimulation may need to be at a different amplitude. ANN-2 periodically produces unusual inputs, such as simultaneous afferent and efferent stimulation commands, which in turn improves the ANN-1's training.

Other implementations of Output Processor can be constructed using a convolutional neural network, auto-encoder, Bayesian optimizer, maximum likelihood estimator, linear support vector machine, and/or classification and regression tree. The same or similar equations and methodologies as discussed in connection with the Input Processor may be used in connection with the output processor.

### Implantable Device

In certain example embodiments, an implantable device includes (e.g., stimulation electrode 0130) is configured to provide long-term therapy to the patient under ambulatory conditions. FIG. 27 shows the configuration of the implantable device 2700 that includes an implant input processor 2702 (which may share any or all of the features of input processor 202), an implant control processor 2704 (which may share any or all of the features of control processor 204), an implant output processor 2706 (which may share any or all of the features of output processor 206), and an implant stim circuit 2708 (which may share any or all of the features of stimulation electrode 0130).

In certain examples, the input signals that are received and/or processed by the input processor 2702 include any or all of the signals that relate to the following: eCAP, accelerometer, gyroscope, heart rate, oxygen saturation, pressure, and electrical impedance. Implant input processor 2702 implements the algorithm that is determined to be the optimal one for processing of the input signals. This algorithm and its parameters were determined during the training phase, as described in the Input Signal Processor section above. Parameters of the algorithm that runs on the Implant Input Processor were learned during the training phase and were customized for the patient who is wearing the implantable device. Output of the Implant Input Processor contains signals indicating the detection of a successful inhale, exhale, an obstructed breath and so on, which is fed into the Implant Control Processor. Implant Control Processor works to maintain a desired respiration rate and or a minimum oxygen saturation. Hence, the Implant Control Processor generates the commands such as Stimulate Efferents and Stimulate Afferents when it is needed. Implant Output Processor generates the parameters that are needed for the generation of the stimulation waveform, including but not limited to, stimulation amplitude, pulse width, stimulation frequency and number of pulses. Again, the Implant Output Processor uses the algorithm and its associated parameters that were determined to be the optimal ones during the training phase.

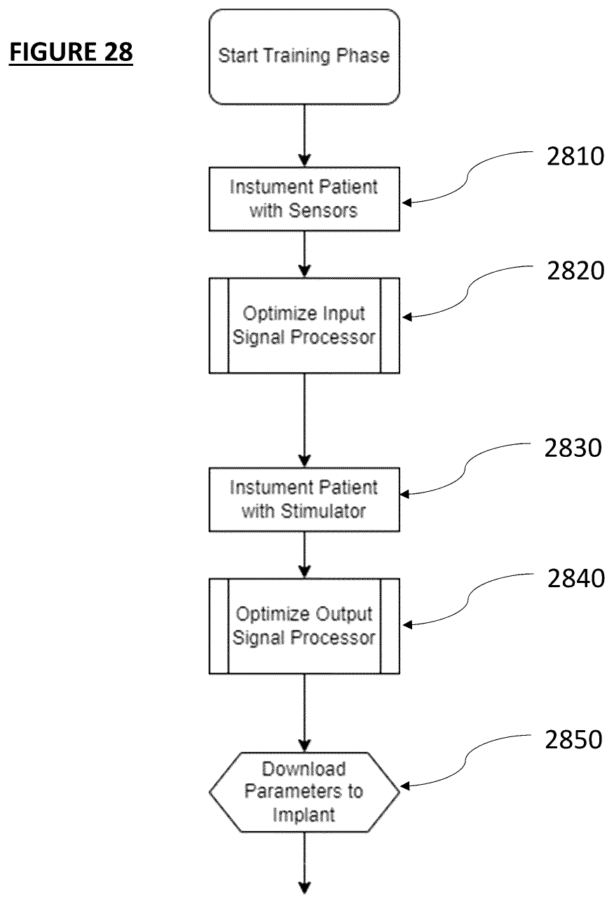

### Description of FIG. 28

In one embodiment, as shown in FIG. 28, the training phase is carried out by instrumenting the patient with external devices 2810, such as sensors, and the optimum algorithm for the input signal processor is determined using external computers 2820. Afterwards, the patient is instrumented with external stimulators 2830 and the output processor is optimized 2840. Finally, the optimum algorithms for the input signal processor and the output processor are downloaded into the implant for ambulatory use 2850. This embodiment offers the advantage of determining which set of sensors would be required by the implant and only implanting them into the patient.

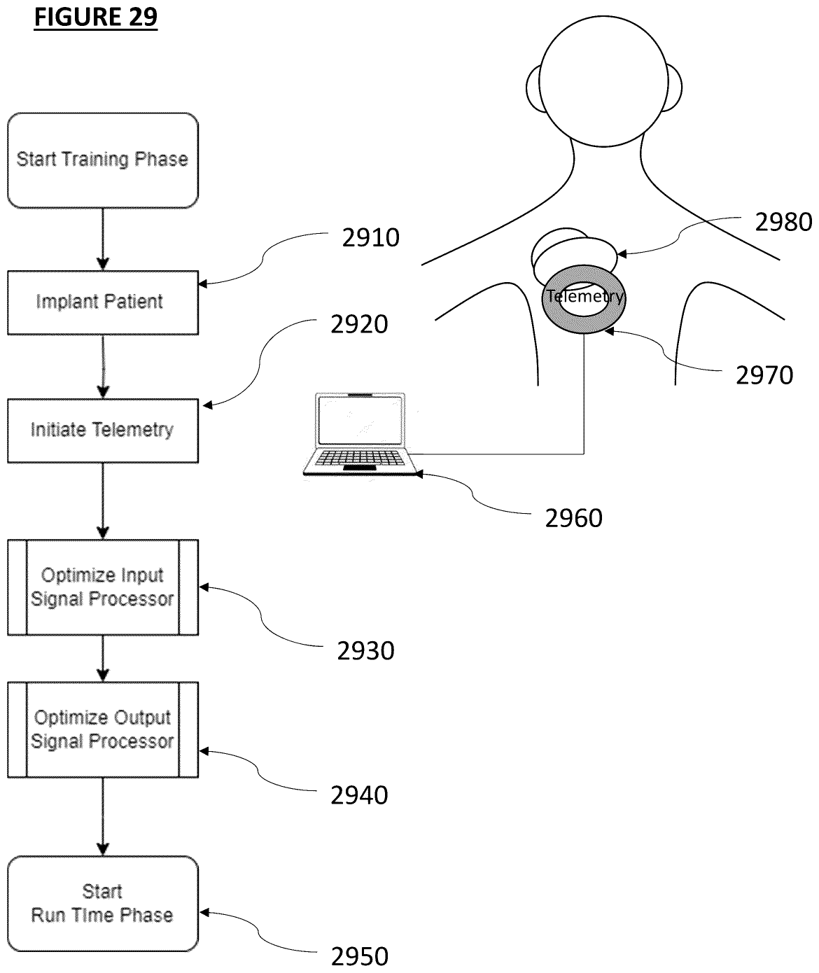

### Description of FIG. 29

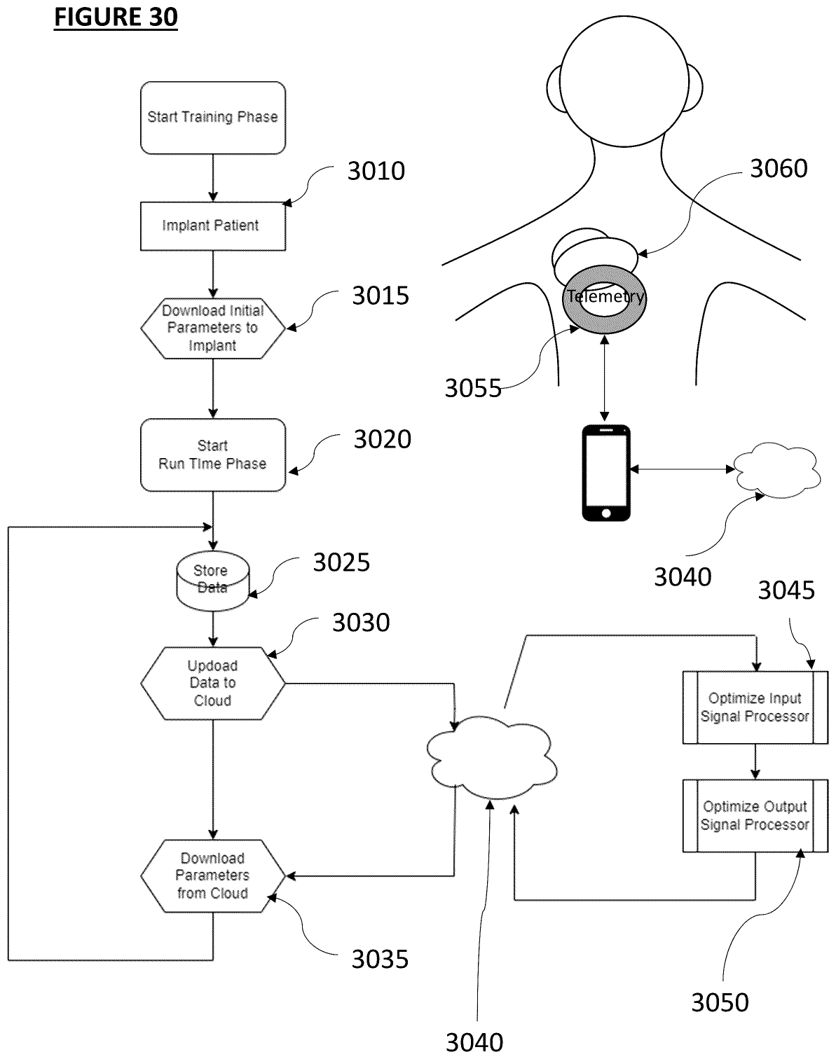

In a different embodiment, as shown in FIG. 29, the training phase is carried out after implanting the implantable device 2980 into the patient 2910. Optimization is carried out by an external computer 2960 that is continuously in communication with the implanted device via telemetry 2920 & 2970. Once the optimum algorithms for the input signal processor and output signal processor are determined by the external computer 2960 where the external computer 2960 continuously receives the sensory signals via telemetry and provides stimulation directives via telemetry to the implant 2980, while carrying out the optimization tasks 2930 & 2940. Once the optimization is completed, the resulting parameter sets are downloaded into the implant via telemetry 2970 for ambulatory use 2950. This embodiment offers the advantage of having the optimization done under situations that closely resemble the use conditions, such as sleep at home. Description of FIG. 30

Yet in another embodiment, as shown in FIG. 30, the training phase is carried out in the cloud 3040 after implanting the implantable device 3060 into the patient. Following the implant, an initial set of parameters are downloaded into the implant 3015 and the run time phase begins 3020. Data that is generated from the sensors and the treatment parameters are stored 3025 and periodically uploaded 3030 into the cloud 3040 where the optimization takes place 3045 & 3050. Subsequently, the parameters of the optimized algorithm are downloaded 3035 and used by the implant 3060. This embodiment offers two advantages: First the optimization occurs in an ongoing manner, and second the optimized set of parameters reside in the cloud, which can be used as the initial set of parameters for future patients.

### Description of FIG. 31

FIG. 31 is a block diagram of an example computing device 3100 (which may also be referred to, for example, as a “computing device,” “computer system,” or “computing system”) according to some embodiments. In some embodiments, the computing device 3100 includes one or more of the following: one or more processors 3102 (which may be referred to as “hardware processors” or individually as a “hardware processor”); one or more memory devices 3104; one or more network interface devices 3106; one or more display interfaces 3108; and one or more user input adapters 3110. Additionally, in some embodiments, the computing device 3100 is connected to or includes a display device 3112. As will be explained below, these elements (e.g., the processors 3102, memory devices 3104, network interface devices 3106, display interfaces 3108, user input adapters 3110, display device 3112) are hardware devices (for example, electronic circuits or combinations of circuits) that are configured to perform various different functions for the computing device 3100. In some embodiments, these components of the computing device 3100 may be collectively referred to as computing resources (e.g., resources that are used to carry out execution of instructions and include the processors (one or more processors 3102), storage (one or more memory devices 3104), and I/O (network interface devices 3106, one or more display interfaces 3108, and one or more user input adapters 3110). In some instances, the term processing resources may be used interchangeably with the term computing resources. In some embodiments, multiple instances of computing device 3100 may be arranged into a distributed computing system.

In some embodiments, each or any of the processors 3102 is or includes, for example, a single- or multi-core processor, a microprocessor (e.g., which may be referred to as a central processing unit or CPU), a digital signal processor (DSP), a microprocessor in association with a DSP core, an Application Specific Integrated Circuit (ASIC), a Field Programmable Gate Array (FPGA) circuit, or a system-on-a-chip (SOC) (e.g., an integrated circuit that includes a CPU and other hardware components such as memory, networking interfaces, and the like). And/or, in some embodiments, each or any of the processors 3102 uses an instruction set architecture such as x86 or Advanced RISC Machine (ARM).

In some embodiments, each or any of the memory devices 3104 is or includes a random access memory (RAM) (such as a Dynamic RAM (DRAM) or Static RAM (SRAM)), a flash memory (based on, e.g., NAND or NOR technology), a hard disk, a magneto-optical medium, an optical medium, cache memory, a register (e.g., that holds instructions), or other type of device that performs the volatile or non-volatile storage of data and/or instructions (e.g., software that is executed on or by processors 3102). Memory devices 3104 are examples of non-transitory computer-readable storage media.

In some embodiments, each or any of the network interface devices 3106 includes one or more circuits (such as a baseband processor and/or a wired or wireless transceiver), and implements layer one, layer two, and/or higher layers for one or more wired communications technologies (such as Ethernet (IEEE 802.3)) and/or wireless communications technologies (such as Bluetooth, WiFi (IEEE 802.11), GSM, CDMA2000, UMTS, LTE, LTE-Advanced (LTE-A), LTE Pro, Fifth Generation New Radio (5G NR) and/or other short-range, mid-range, and/or long-range wireless communications technologies such as Zigby or any custom wireless communication protocol). Transceivers may comprise circuitry for a transmitter and a receiver. The transmitter and receiver may share a common housing and may share some or all of the circuitry in the housing to perform transmission and reception. In some embodiments, the transmitter and receiver of a transceiver may not share any common circuitry and/or may be in the same or separate housings.

In some embodiments, data is communicated over an electronic data network. An electronic data network includes implementations where data is communicated from one computer process space to computer process space and thus may include, for example, inter-process communication, pipes, sockets, and communication that occurs via direct cable, cross-connect cables, fiber channel, wired and wireless networks, and the like. In certain examples, network interface devices 3106 may include ports or other connections that enable such connections to be made and communicate data electronically among the various components of a distributed computing system.

In some embodiments, each or any of the display interfaces 3108 is or includes one or more circuits that receive data from the processors 3102, generate (e.g., via a discrete GPU, an integrated GPU, a CPU executing graphical processing, or the like) corresponding image data based on the received data, and/or output (e.g., a High-Definition Multimedia Interface (HDMI), a DisplayPort Interface, a Video Graphics Array (VGA) interface, a Digital Video Interface (DVI), or the like), the generated image data to the display device 3112, which displays the image data. Alternatively, or additionally, in some embodiments, each or any of the display interfaces 3108 is or includes, for example, a video card, video adapter, or graphics processing unit (GPU).

In some embodiments, each or any of the user input adapters 3110 is or includes one or more circuits that receive and process user input data from one or more user input devices (not shown in FIG. 31) that are included in, attached to, or otherwise in communication with the computing device 3100, and that output data based on the received input data to the processors 3102. Alternatively, or additionally, in some embodiments each or any of the user input adapters 3110 is or includes, for example, a PS/2 interface, a USB interface, a touchscreen controller, or the like; and/or the user input adapters 3110 facilitates input from user input devices (not shown in FIG. 31) such as, for example, a keyboard, mouse, trackpad, touchscreen, etc.

In some embodiments, the display device 3112 may be a Liquid Crystal Display (LCD) display, Light Emitting Diode (LED) display, or other type of display device. In embodiments where the display device 3112 is a component of the computing device 3100 (e.g., the computing device and the display device are included in a unified housing), the display device 3112 may be a touchscreen display or non-touchscreen display. In embodiments where the display device 3112 is connected to the computing device 3100 (e.g., is external to the computing device 3100 and communicates with the computing device 3100 via a wire and/or via wireless communication technology), the display device 3112 is, for example, an external monitor, projector, television, display screen, etc.

In various embodiments, the computing device 3100 includes one, or two, or three, four, or more of each or any of the above-mentioned elements (e.g., the processors 3102, memory devices 3104, network interface devices 3106, display interfaces 3108, and user input adapters 3110). Alternatively, or additionally, in some embodiments, the computing device 3100 includes one or more of: a processing system that includes the processors 3102; a memory or storage system that includes the memory devices 3104; and a network interface system that includes the network interface devices 3106. Alternatively, or additionally, in some embodiments, the computing device 3100 includes a system-on-a-chip (SoC) or multiple SoCs, and each or any of the above-mentioned elements (or various combinations or subsets thereof) is included in the single SoC or distributed across the multiple SoCs in various combinations. For example, the single SoC (or the multiple SoCs) may include the processors 3102 and the network interface devices 3106; or the single SoC (or the multiple SoCs) may include the processors 3102, the network interface devices 3106, and the memory devices 3104; and so on. The computing device 3100 may be arranged in some embodiments such that: the processors 3102 include a multi or single-core processor; the network interface devices 3106 include a first network interface device (which implements, for example, WiFi, Bluetooth, NFC, etc.) and a second network interface device that implements one or more cellular communication technologies (e.g., 3G, 4G LTE, CDMA, etc.); the memory devices 3104 include RAM, flash memory, or a hard disk. As another example, the computing device 3100 may be arranged such that: the processors 3102 include two, three, four, five, or more multi-core processors; the network interface devices 3106 include a first network interface device that implements Ethernet and a second network interface device that implements WiFi and/or Bluetooth; and the memory devices 3104 include a RAM and a flash memory or hard disk.

The hardware configurations shown in FIG. 31 and described above are provided as examples, and the subject matter described herein may be utilized in conjunction with a variety of different hardware architectures and elements. For example: in many of the Figures in this document, individual functional/action blocks are shown; in various embodiments, the functions of those blocks may be implemented using (a) individual hardware circuits, (b) using an application specific integrated circuit (ASIC) specifically configured to perform the described functions/actions, (c) using one or more digital signal processors (DSPs) specifically configured to perform the described functions/actions, (d) using the hardware configuration described above with reference to FIG. 31, (e) via other hardware arrangements, architectures, and configurations, and/or via combinations of the technology described in (a) through (e).

### Selected Terminology

The elements described in this document include actions, features, components, items, attributes, and other terms. Whenever it is described in this document that a given element is present in “some embodiments,” “various embodiments,” “certain embodiments,” “certain example embodiments, “some example embodiments,” “an exemplary embodiment,” “an example,” “an instance,” “an example instance,” or whenever any other similar language is used, it should be understood that the given element is present in at least one embodiment, though is not necessarily present in all embodiments. Consistent with the foregoing, whenever it is described in this document that an action “may,” “can,” or “could” be performed, that a feature, element, or component “may,” “can,” or “could” be included in or is applicable to a given context, that a given item “may,” “can,” or “could” possess a given attribute, or whenever any similar phrase involving the term “may,” “can,” or “could” is used, it should be understood that the given action, feature, element, component, attribute, etc. is present in at least one embodiment, though is not necessarily present in all embodiments.

Terms and phrases used in this document, and variations thereof, unless otherwise expressly stated, should be construed as open-ended rather than limiting. As examples of the foregoing: “and/or” includes any and all combinations of one or more of the associated listed items (e.g., a and/or b means a, b, or a and b); the singular forms “a”, “an”, and “the” should be read as meaning “at least one,” “one or more,” or the like; the term “example”, which may be used interchangeably with the term embodiment, is used to provide examples of the subject matter under discussion, not an exhaustive or limiting list thereof; the terms “comprise” and “include” (and other conjugations and other variations thereof) specify the presence of the associated listed elements but do not preclude the presence or addition of one or more other elements; and if an element is described as “optional,” such description should not be understood to indicate that other elements, not so described, are required.

As used herein, the term “non-transitory computer-readable storage medium” includes a register, a cache memory, a ROM, a semiconductor memory device (such as D-RAM, S-RAM, or other RAM), a magnetic medium such as a flash memory, a hard disk, a magneto-optical medium, an optical medium such as a CD-ROM, a DVD, or Blu-Ray Disc, or other types of volatile or non-volatile storage devices for non-transitory electronic data storage. The term “non-transitory computer-readable storage medium” does not include a transitory, propagating electromagnetic signal.

The claims are not intended to invoke means-plus-function construction/interpretation unless they expressly use the phrase “means for” or “step for.” Claim elements intended to be construed/interpreted as means-plus-function language, if any, will expressly manifest that intention by reciting the phrase “means for” or “step for”; the foregoing applies to claim elements in all types of claims (method claims, apparatus claims, or claims of other types) and, for the avoidance of doubt, also applies to claim elements that are nested within method claims. Consistent with the preceding sentence, no claim element (in any claim of any type) should be construed/interpreted using means plus function construction/interpretation unless the claim element is expressly recited using the phrase “means for” or “step for.”

Whenever it is stated herein that a hardware element (e.g., a processor, a network interface, a display interface, a user input adapter, a memory device, or other hardware element), or combination of hardware elements, is “configured to” perform some action, it should be understood that such language specifies a physical state of configuration of the hardware element(s) and not mere intended use or capability of the hardware element(s). The physical state of configuration of the hardware elements(s) fundamentally ties the action(s) recited following the “configured to” phrase to the physical characteristics of the hardware element(s) recited before the “configured to” phrase. In some embodiments, the physical state of configuration of the hardware elements may be realized as an application specific integrated circuit (ASIC) that includes one or more electronic circuits arranged to perform the action, or a field programmable gate array (FPGA) that includes programmable electronic logic circuits that are arranged in series or parallel to perform the action in accordance with one or more instructions (e.g., via a configuration file for the FPGA). In some embodiments, the physical state of configuration of the hardware element may be specified through storing (e.g., in a memory device) program code (e.g., instructions in the form of firmware, software, etc.) that, when executed by a hardware processor, causes the hardware elements (e.g., by configuration of registers, memory, etc.) to perform the actions in accordance with the program code.

A hardware element (or elements) can therefore be understood to be configured to perform an action even when the specified hardware element(s) is/are not currently performing the action or is not operational (e.g., is not on, powered, being used, or the like). Consistent with the preceding, the phrase “configured to” in claims should not be construed/interpreted, in any claim type (method claims, apparatus claims, or claims of other types), as being a means plus function; this includes claim elements (such as hardware elements) that are nested in method claims.

### Additional Applications of Described Subject Matter

Although process steps, algorithms, or the like, including without limitation with reference to FIGS. 28-30, may be described or claimed in a particular sequential order, such processes may be configured to work in different orders. In other words, any sequence or order of steps that may be explicitly described or claimed in this document does not necessarily indicate a requirement that the steps be performed in that order; rather, the steps of processes described herein may be performed in any order possible. Further, some steps may be performed simultaneously (or in parallel) despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary, and does not imply that the illustrated process is preferred.

Although various embodiments have been shown and described in detail, the claims are not limited to any particular embodiment or example. None of the above description should be read as implying that any particular element, step, range, or function is essential. All structural and functional equivalents to the elements of the above-described embodiments that are known to those of ordinary skill in the art are expressly incorporated herein by reference and are intended to be encompassed. Moreover, it is not necessary for a device or method to address each and every problem sought to be solved by the present invention, for it to be encompassed by the invention. No embodiment, feature, element, component, or step in this document is intended to be dedicated to the public.

1. A system for automated sensing, control, and stimulation to treat sleep apnea in a patient, the system comprising:
one or more sensors configured to generate a signal based on at least one physiological characteristic of a patient;
an input processor that is configured to:
(a) determine, based on the generated signal, a set of input parameters out of multiple possible sets of input parameters to use in connection with detection of respiratory activity in the patient, and
(b) determine, for each corresponding input parameter in the determined set of input parameters, a parameter value for the corresponding input parameter;
a control processor configured to prescribe, based on the parameter values determined by the input processor for each of the input parameters in the set of input parameters, an action, out of a plurality of possible actions, to be taken for the patient;
an output processor that is configured to:
(1) determine, based on the determined action, one or more stimulation parameters to use out of a plurality of possible stimulation parameters;
(2) determine, for each respective one of the one or more stimulation parameters, a value for the respective stimulation parameter to be used for modulation of respiratory activity of the patient; implantable hardware to carry out the sensing, control, and stimulation functions using the parameter values in real time.

2. The system of claim 1, wherein the set of sensors includes one or more of nerve action potential sensor, an electrical impedance sensor, an accelerometer, a gyroscope, an oxygen saturation sensor, and a pressure sensor.

3. The system of claim 1, wherein the input signal processor includes one or more of an artificial neural network (ANN), convolutional neural network, generative adversarial network (GAN), auto-encoder, Bayesian optimizer, maximum likelihood estimator, linear support vector machine, and classification and regression tree.

4. The system of claim 1, wherein the input signal processor is trained to extract values from respiration waveforms by adjusting the parameter values of a model that is used to detect respiratory activity.

5. The system of claim 4, wherein the values that are extracted as associated with are one or more of arousal, inhalation, exhalation, apnea, hypopnea, and apnea-hypopnea index.

6. The system of claim 1, wherein the control processor includes is a decision algorithm that maintains a minimum respiratory rate or minimum oxygen saturation by driving the output processor.

7. The system of claim 1, wherein the output processor includes of one or more of an artificial neural network (ANN), convolutional neural network, generative adversarial network (GAN), auto-encoder, Bayesian optimizer, maximum likelihood estimator, linear support vector machine, and classification and regression tree.

8. The system of claim 1, wherein the output processor is trained to generate the values for electrical stimulation waveforms to modulate the physiological system of the patient by adjusting parameter values of the stimulator.

9. The system of claim 1, wherein the plurality of possible stimulation parameters include stimulation amplitude, stimulation pulse width, stimulation frequency, number of pulses in a train, and train timing.

10. The system of claim 1, wherein the action includes one or more of: 1) stimulate efferents, 2) stimulate afferents; 3) set duration; 4) turn on; and/or 5) turn off.

11. The system of claim 1, wherein the set of input parameters and the one or more stimulation parameters are determined before the implantable hardware is implanted in the patient and/or without the use of the implantable device.

12. The system of claim 1, wherein the set of input parameters and the one or more stimulation parameters are determined before the implantable hardware is implanted in the patient and with the use of the implantable device.

13. The system of claim 1, wherein the determined set of input parameters and the determined one or more stimulation parameters are configured to be downloaded to the implantable device.

14. The system of claim 1, wherein the implantable device utilizes the determined set of input parameters and the determined one or more stimulation parameters for the generation and delivery of the therapy to the patient.

15. The system of claim 1 is further configured to determine and update the determined set of input parameters and the determined one or more stimulation parameters during the use of the implantable device to deliver the therapy while optimizing an objective function.

16. The system of claim 1, wherein the determined set of input parameters and the determined one or more stimulation parameters are configured to be communicated with other devices through cloud or local storage systems.

17. The system of claim 1, wherein parameters are configured to be received from one or more remote computing systems.
